SEQUENCE LISTING (5 sequences)

SEQ ID NO: 1 - protein, 16 aa, Artificial
  source Salmo salar and Oncorynchus mykiss
TYFAGAAADVLEVGVR

SEQ ID NO: 2 - protein, 9 aa, Artificial
  source Melanogrammus aeglefinus, Gadus morhua and Pagrus major
DLGLAYTEK

SEQ ID NO: 3 - protein, 13 aa, Artificial
  source Reinhardtius hippoglossoides, Pagrus major, Morone amiricana, Oncorynchus mykiss and Verasper moseri
FFGQEIAFANIDK

SEQ ID NO: 4 - protein, 13 aa, Artificial
  source Epinephelus coioides, Verasper moseri, Morone amiricana, Oncorynchus mykiss and Reinhardtius hippoglossoides
EIVLLGYGTMISK

SEQ ID NO: 5 - protein, 8 aa, Artificial
  source Melanogrammus aeglefinus and Gadus morhua
YESFAVAR

(19) United States
(12) Patent Application Publication
(10) Pub. No.: US 2009/0011447 A1
(43) Pub. Date: Jan. 8, 2009

(54) QUANTIFICATION OF VITELLOGENIN

(76) Inventors: Joseph H. Banoub, St. John's (CA); Alejandro Cohen, St. John's (CA); Atef Mansour, St. John's (CA)

Correspondence Address: THE FARRELL LAW FIRM, P.C., 333 EARLE OVINGTON BOULEVARD, SUITE 701, UNIONDALE, NY, 11553, US

(21) Appl. No.: 11/754,002

(22) Filed: May 25, 2007

Related U.S. Application Data
(60) Provisional application No. 60/808,538, filed on May 26, 2006.
(60) Provisional application No. 60/876,476, filed on Dec. 22, 2006.

(30) Foreign Application Priority Data
May 25, 2006 (CA) 2,549,079
Dec. 21, 2006 (CA) 2,575,507

(51) Int. Cl. C12Q 1/56

(52) U.S. Cl. 435/13

(57) ABSTRACT

The present invention is directed to a simple method for absolute quantification of plasma vitellogenin from two or more different fish species such as Rainbow trout and Atlantic salmon, or Atlantic cod and haddock. In the case of Rainbow trout and Atlantic salmon, plasma samples obtained from control and β-estradiol induced fish were digested with trypsin. A characteristic ‘signature peptide’ was selected and analyzed by high performance liquid chromatography coupled to an electrospray quadrupole-time-of-flight tandem mass spectrometer, using a deuterated homologue peptide as an internal standard. The hybrid tandem mass spectrometer was operated in a ‘pseudo’ selected reaction monitoring mode by which three diagnostic product ions were monitored for identification and quantification purposes. The reproducibility (coefficient of variation ˜5%) and sensitivity (limit of quantification of 0.009 mg/mL) achieved by this simple assay allow it to be considered as an alternative to immunological assays. In the case of Atlantic cod and haddock, the amino acid sequence of the vitellogenin protein has not yet been determined, but, the Atlantic cod vitellogenin has been characterized using a ‘bottom-up’ mass spectrometric approach. Vitellogenin synthesis was induced ‘in vivo’ with β-Estradiol, and subjected to trypsin digestion for characterization by matrix-assisted laser desorption/ionization-Quadrupole-Time-of-flight tandem mass spectrometry. A peptide mass fingerprint was obtained and ‘de novo’ sequencing of the most abundant tryptic peptides was performed by low energy collision induced dissociation-tandem mass spectrometry. Thus, the sequences of various tryptic peptides have been elucidated. It has also been determined that Atlantic cod vitellogenin shares a series of common peptides with the two different known vitellogenin sequences of Haddock, a closely related species. There are also disclosed novel isolated signature peptides, namely Thr-Tyr-Phe-Ala-Gly-Ala-Ala-Ala-Asp-Val-Leu-Glu-Val-Gly-Val-Arg, Asp Leu Gly Leu Ala Tyr Thr Glu Lys, Phe Phe Gly Gln Glu Ile Ala Asn Ile Asp Lys, Glu Ile Val Leu Leu Gly Tyr Gly Thr Met Ile Ser Lys and Tyr Glu Ser Phe Ala Val Ala Arg.

### PRIORITY

This application claims priority under 35 U.S.C. § 119 to applications filed in the Canadian Intellectual Property Office on May 25, 2006 and Dec. 21, 2006, assigned Serial Nos. 2,549,079 and 2,575,507, respectively, as well as to U.S. provisional applications filed on May 26, 2006 and Dec. 22, 2006, assigned Ser. Nos. 60/808,538 and 60/876,476, respectively, the contents of each which are incorporated herein by reference.

### FIELD OF THE INVENTION

This invention relates to various peptides which have been isolated and which are useful as signature peptides for determination of plasma vitellogenin in various species of fish; as well, this invention relates to a method for absolute quantification of plasma vitellogenin from various fish species such as related species exemplified by Rainbow trout and Atlantic salmon, and Atlantic cod and haddock.

### BACKGROUND OF THE INVENTION

Vitellogenin (Vtg), a phosphoglycolipoprotein, is synthesized in the liver of oviparous animals in response to circulating estrogens. Its molecular weight may vary from 200 to 500 KDa, depending on the species. In sexually mature females, Vtg is secreted into the blood stream and is incorporated into the oocyte by receptor mediated endocytosis, where it is further cleaved. The exact functions of these proteins are still uncertain, although it is generally accepted that these proteins are finally hydrolyzed into a free amino acid pool, which serves as the main nutritional source for the developing embryo.^{1,2,3}

Vtg was first described in insects by Pan et al in 1969.⁴ Since then, it has been the focus of much research, especially in the area of embryology. In the past decade, there has been a renewed interest in Vtg due to its potential use as an environmental biomarker.^{5,6,7}

Usually, Vtg is undetectable in males under physiological conditions. However, some in-vitro and field studies have demonstrated high Vtg plasma levels in male vertebrates exposed to certain xenobiotic endocrine disruptors with estrogen-mimicking activity.^{8,9}

Investigations have been conducted of the Vtg levels in different fish species for their use in the fisheries and aquaculture industry as prospective indicators for assessing the sexual reproductive status and maturity of fish stocks.^{10,11,12}

The prior art teaches a wide spectrum of analytical methods to determine the quantification of Vtg levels. Indirect measurement of Vtg has been achieved by calorimetrically determining the alkaline-labile phosphorous content of the fish plasma.¹³ More recently, a number of immunoassays, such as the competitive enzyme-linked immunosorbent assays (ELISA)^{6,14,15} and radioimmunoassays (RIA)^{16,17,18} have been developed. Similarly, Vtg mRNA levels have been assayed¹⁹ although the use of liver homogenate samples in this technique are prohibitive for large scale animal monitoring.

Advances in mass spectrometry (MS) have provided researchers with powerful quantitative techniques in the area of proteomics.²⁰ Quantification of proteins can be achieved by using either a relative or an absolute technique. Relative quantification is usually sufficient in those studies which focus on differential protein expression between control and experimental groups. Different strategies have been adopted for this type of quantification. For example, the ‘signature peptide’ approach has availed of the isotopic labeling of specific functional groups in proteolytic peptides by means of derivatization agents.^{21,22,23} Similar techniques use more sophisticated isotope-coded affinity tags^{24,25,26,27,28}, many of which are commercially available. Alternatively, relative quantification can be done by ¹⁸O labeling of enzymatically digested proteins with H₂¹⁸O^{29,30} and by measuring accurate mass and time tags.^{31,32,33,34,35,36,37,38,39}

Absolute protein quantification requires a different approach. The rationale for one approach is that peptides generated by the digestion of complex mixtures may be used as analytical surrogates for the protein from which they are derived. Such an approach is based on the fact that single peptides are in many cases easier to separate and identify than the intact proteins. Furthermore, the synthesis of custom made peptide standards and internal standards is easier than the isolation and purification of the intact proteins for calibration purposes.²³ The internal standards usually consist of homologues of the analyzed peptides in which one or more atoms have been replaced by stable isotopes (e.g. ¹³C, ¹⁵N, ²H etc). Variations of this approach have been applied to different proteins: Fathead minnow (Pimephales promelas) Vtg, rhodopsin, apolipoprotein A-1 and a hemoglobin β-chain derived glycated hexapeptide have been quantified using triple quadrupole mass spectrometers.^{40,41,42,43} Other absolute quantification studies in the area of proteomics have been described and are known as AQUA (Absolute QUAantification)^{44,45,46} strategy and SISCAPA⁴⁷ (Stable Isotope Standards and Capture by Anti-Peptide Antibodies).

### SUMMARY OF THE INVENTION

The present invention discloses novel isolated signature peptides such as Thr-Tyr-Phe-Ala-Gly-Ala-Ala-Ala-Asp-Val-Leu-Glu-Val-Gly-Val-Arg, and Asp Leu Gly Leu Ala Tyr Thr Glu Lys.

Other peptides include Phe Phe Gly Gln Glu Ile Ala Asn Ile Asp Lys, Glu Ile Val Leu Leu Gly Tyr Gly Thr Met Ile Ser Lys and Tyr Glu Ser Phe Ala Val Ala Arg.

According to another aspect of the present invention, there is provided a method of quantification of fish plasma Vitellogenin using signature peptides in a mass spectrometry comprising the steps of selecting a desired signature peptide from at least one fish species, and determining the presence or absence of the selected signature peptide in said fish species by liquid chromatography in combination with mass spectrometry.

In the present invention, the method is most desirably employed using a plurality of fish species where such plurality of species has a common signature peptide so that with the method of the present invention, the use of the mass spectrometric step is capable of determining the plasma Vtg levels of two or more species of fish such as, e.g., Rainbow trout and Atlantic salmon, or Atlantic cod and haddock. The method of the present invention thus permits the Vtg level of two or more fish species to be simultaneously quantified using the ‘signature peptide’ approach described herein.

The method of the present invention provides a simple approach by using a single step ‘one pot’ trypsin digestion of the serum, without the need of protein isolation and purification.

With respect to the method of the present invention involving the species of Atlantic cod and haddock, a preferred embodiment involves the steps of initially separating Vtg from the serum proteins of P-Estradiol injected Atlantic cod by sodium dodecylsulphate polyacrylamide gel electrophoresis (SDS-PAGE). The band of interest was excised and subjected to ‘in-gel’ enzymatic digestion with trypsin. The tryptic peptides obtained were extracted and analyzed by matrix-assisted laser desorption/ionization-Quadrupole-Time-of-flight mass spectrometry (MALDI-QqToF-MS) and tandem mass spectrometry (MALDI-QqToF-MS/MS). The peptide mass fingerprint (PMF) obtained from the MALDI-QqToF-MS experiments interestingly showed a series of common peptides with those of Haddock (Melanogrammus aeglefinus) Vtg, a closely related species.

The sequence of some of these peptides was confirmed by tandem mass spectrometry. Moreover, some of the matching peptides could be distinctively assigned to the two forms of Haddock Vtg described recently by Reith et al.⁶⁴ This strongly suggests that Atlantic cod would also express at least two forms of Vtg.

Other sources or species of fish may also include Greenland halibut (Reinhardtius hippoglossoides), Red seabream (Pagrus major), White perch (Morone amiricana), Barfin flounder (Verasper moseri) and Orange-spotted grouper (Epinephelus coioides).

### BRIEF DESCRIPTION OF THE DRAWINGS

Further aspects and advantages will become apparent from the following detailed description according to preferred embodiments with reference to the drawings:

In the following drawings, FIGS. 1 to 6 illustrate details relating to the Rainbow trout and Salmon species; FIGS. 7 to 11 relate to results dealing with Atlantic cod and haddock.

FIG. 1 is a schematic outline of the method of the present invention showing that blood is extracted from fish and spun to obtain unprocessed plasma, following which labeled peptide is added to the trypsin digestion, and subsequently analyzed by HPLC-QqToF-MS/MS.

FIG. 2 is the spectra of doubly charged signature peptide at m/z 819.9, and FIG. 2A illustrates the corresponding deuterated isotopic homologue at m/z 824.4. FIG. 2B shows an inset of the expected b and y-type peptide ions. The product ions observed in the spectra are highlighted with boxes. The Y₉, Y₁₀ and Y₁₂ diagnostic ions used for identification and quantification purposes during the ‘pseudo’ SRM-MS/MS are labeled accordingly. A* stands for L-Alanine-3,3,3-D₃.

FIG. 3 comprising sub-FIGS. 3A, 3B, 3C and 3D illustrate the details of a plasma digested sample from Rainbow trout analyzed in both HPLC-QqToF-MS (FIGS. 3A and 3B) and MS/MS (FIGS. 3C and 3D) modes for comparison purposes. More particularly, FIG. 3A shows the complex TIC chromatogram acquired in full scan mode, while FIG. 3B shows the extracted ion chromatogram (EIC) from FIG. 3A for the ions at m/z 819.9. In FIG. 3C, there is illustrated the results of a TIC chromatogram, obtained in the ‘pseudo’ SRM scan of the m/z 819.9 precursor. FIG. 3D shows the superimposed EICs obtained from the Y₉, Y₁₀ and Y₁₂ diagnostic ions. The inset shows an expanded scale of the peak showing the traces obtained from each product.

FIG. 4 comprises FIGS. 4A through 4E in which FIG. 4A shows the ‘pseudo’ SRM chromatogram obtained from HPLC-QqToF-MS/MS analysis of a Rainbow trout plasma digest sample while FIGS. 4B and 4C show the mass range windows used for the extracted chromatograms shown in FIGS. 4D and 4E, respectively.

FIG. 5 is a full scan ToF-MS of the labeled signature peptide directly infused into the mass spectrometer. The peak at m/z 824.5 corresponds to the [M+2H]²⁺ species. It is to be noted from this Figure that there is the absence of peaks at m/z 819.9 corresponding to [M+2H]²⁺ of the non-labeled peptide.

FIG. 6 comprises FIGS. 6A and 6B which show overlaid extracted ion chromatograms of the Y₉, Y₁₀ and Y₁₂ diagnostic ions obtained from the ‘pseudo’ SRM-MS/MS analysis for the same fish prior to (FIG. 6A) and following β-estradiol injection (FIG. 6B). Both figures show the traces corresponding to the signature peptide (SP) and the internal standard (IS). The inset shows an expanded scale of the same chromatograms.

FIG. 7 illustrates the results of a control (C) and the experimental (E) serum samples run on an SDS-PAGE and stained with Coomassie blue. Note the intense induced Vtg band on the experimental samples.

FIG. 8, showing two spectra graphs, is based on the results obtained by MALDI-MS of the ‘in-gel’ trypsin digestion of Atlantic cod vitellogenin. Both spectra correspond to the same acquisition. The axes have been cut to improve peak visualization. The top and bottom spectra correspond to an m/z range of 800-1800 and 1700-3000 respectively.

FIG. 9 is the product ion spectrum of [M+H]⁺ ion at m/z 1089.54. The expected fragmentation pattern is shown in the inset. The observed diagnostic B and Y peptide product ions are outlined in boxes.

FIG. 10 is the product ion spectrum of [M+H]⁺ ion at m/z 1106.58. The expected fragmentation pattern is shown in the inset. The observed diagnostic B and Y peptide product ions are outlined in boxes.

FIG. 11, comprising FIGS. 11A, 11B and 11C, illustrates the product ion spectra of [M+H]+precursor ions at m/z 942.49, 1009.53 and 1124.13. The expected fragmentation pattern is shown in the inset of this Figure. The observed diagnostic B and Y peptide product ions are outlined in boxes.

### DESCRIPTION OF THE PREFERRED EMBODIMENTS

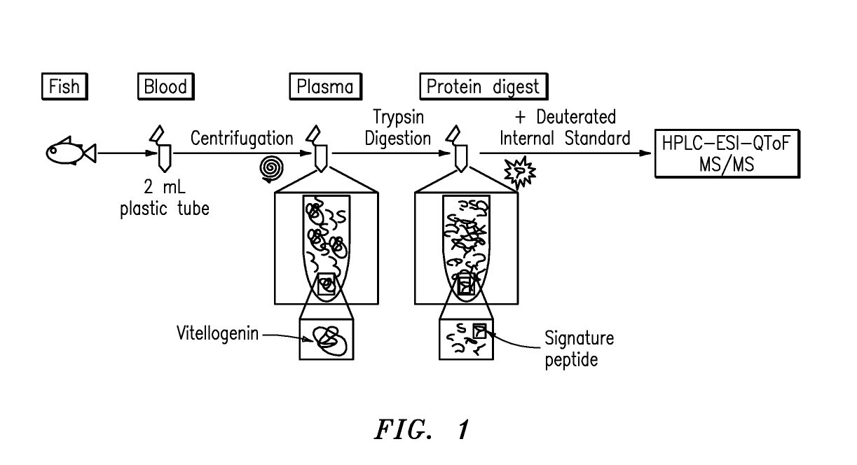

The following description relates to a particularly preferred embodiment, where two fish species are employed for use in the method of the present invention. In this embodiment, there is employed a characteristic ‘signature’ proteolytic peptide, common to both Rainbow trout and Atlantic salmon Vtg was monitored by high performance liquid chromatography (HPLC) coupled to an electrospray ionization quadrupole-time-of-flight (ESI-QqToF-MS/MS) tandem mass spectrometer. A diagram of the work scheme is summarized in FIG. 1. A different approach has also been proposed for this protein using HPLC-MALDI-MS.⁴⁸

Past research has focused on the characterization of Rainbow trout and Atlantic salmon Vtg, using matrix-assisted laser desorption/ionization-time-of-flight (MALDI-ToF) and ESI-low energy collision induced dissociation (CID)-MS/MS.^{10,11} Similar experiments have also been carried out for Atlantic cod.¹² These experiments can provide the information necessary for the selection of these diagnostic ‘signature peptides’.

Quantification by mass spectrometry has commonly been performed on triple quadrupole (QqQ) tandem mass spectrometers. The mass spectrometry instruments have shown exceptional results when operated in Selected Reaction Monitoring (SRM) mode. Alternatively, in the present invention, the use of the hybrid QqToF-MS/MS instrument for quantification purposes^{49,50} can be employed where desired. Although the QqToF-MS/MS configuration has already been used for quantitative purposes^{51,52,53,54}, no research has yet been published using this system for the quantification of such a complex protein.

This method, developed by adjusting and optimizing the tuning parameters of the QqToF analyzer, is termed ‘pseudo’ SRM herein. The CID-MS/MS method was programmed to operate in a mode that monitors the products of both the ‘signature peptide’ and its deuterated isotopic homologue, used as an internal standard.

### Experimental Procedures

Urea, dithiothreitol, aprotinin, tricaine methanesulfonate, NH4HCO₃, acetic acid, acetone, β-estradiol and trypsin were employed herein, and obtained from Sigma (St. Louis, Mo., USA). Acetonitrile was obtained from Anachemia (Canada). For the chromatographic separations, three mobile phases containing H₂O, acetonitrile and acetic acid were prepared in the following proportions: Solvent A (5:95:0.1), Solvent B (70:30:0.1) and Solvent C (95:5:0.1). The standard peptide (purity >95%) and its deuterated isotopic homologue (TYFAGA*A*A*DVLEVGVR, purity >95% with A* being L-Alanine-3,3,3-d3) to be used as internal standard were acquired from Genemed Synthesis (San Francisco, Calif., USA). Solutions of the standard peptide were prepared at concentrations of 0.05, 0.10, 0.25 and 0.50 μg/mL each containing 0.1 μg/mL of the internal standard. These calibration solutions were prepared in Solvent B.

Vtg Induction with β-Estradiol

### Part A

Thirteen juvenile Rainbow trout and ten mature Atlantic salmon were kept in two 60 gallon tanks with running fresh and sea water, respectively. A 12 hour light-darkness cycle was maintained during the experiment. After one month of acclimatization, experimental fish were injected intraperitoneally four times, at one week intervals, with a dose of 10 mg of β-estradiol per kilogram of body weight using a 10 mg/mL solution in oil:acetone (9:1). Control fish were injected with the oil:acetone solution only.

One week before the first injection and one week after the last injection, fish were anesthetized using tricaine methanesulfonate (TMS) and blood samples were collected from the caudal vein using heparinized syringes and transferred in 1 mL portions into plastic tubes kept in ice. Samples were centrifuged at 3000 rpm for 5 minutes at 0° C. Plasma was separated and kept frozen at −80° C. until further analysis.

### Part B

Twelve mature female Atlantic cod fish were kept in a 60 gallon tank with a running sea water supply and fed, ad libitum. A 12 hour light-darkness cycle was maintained during the experiment. After one month of acclimatization, experimental fish were injected intraperitoneally four times, at one week intervals, a dose of 10 mg of β-estradiol per kilogram of fish body weight using a 10 mg/ml solution in oil:acetone (9:1). Control fish were injected only with the oil:acetone solution.

One week after the last injection, fish were anesthetized using tricaine methanesulfonate (TMS) and bled. Blood samples were collected from the caudal vein using heparinized syringes and transferred in 1 mL portions into plastic tubes kept in ice. Twenty microliters of a 4 TIU aprotinin solution were added to each sample, homogenized and centrifuged at 3000 rpm for 5 minutes at 0° C. Plasma was separated and kept frozen at −80° C. until further analysis.

### Enzymatic Digestion of Plasma.

### For Part A

Five μL of plasma were mixed with 100 μL of denaturing buffer (8M urea, 4 mM dithiothreitol) and heated at 65° C. Then, 300 μL of a 50 mM solution of NH₄HCO₃ were added, followed by 95 μL of a 20 μg/mL trypsin solution. Digestion was carried out at 38° C. for 24 hours. Aliquots of 100 μL of the digested samples were mixed with 380 μL of solvent B (H₂O: acetonitrile: acetic acid 70:30:0.1) and spiked with 20 μL of 5 μg/mL labeled standard solution. Finally, the samples were passed through 0.45 μm filters (Millipore Billerica Mass., USA, Cat. No. SJHV004NS) prior to injection into the HPLC.

### For Part B

### “In-Gel” Trypsin Digestion

Separation of Vtg by SDS-PAGE and subsequent “in-gel” digestion was performed following the procedure described in detail by Kinter et al. with minor modifications⁶⁵. Five microliters of thawed plasma from both experimental and control fish were mixed and loaded onto a 6% acrylamide gel and run for one hour at 180 V.

The gel was stained using the colloidal Coomassie blue protocol. The band corresponding to the induced Vtg was easily recognized by comparison of the protein profile from both control and experimental fish. This band was cut into 1 mm³ pieces using surgical blades and placed into 2 mL plastic vials for enzymatic digestion. An unstained piece of gel from one corner of the gel slab was simultaneously processed for use as a blank. Samples were unstained, reduced with 10 mM dithiothreitol and alkylated with 100 mM iodoacetamide.

Enzymatic digestion was performed by adding 30 μL of a 20 ng/mL solution of trypsin and letting the tubes stand in a water bath at 37° C. for 24 hours. The tryptic peptides were extracted in three steps using 30 uL of a 50% v/v solution of acetonitrile containing 5% v/v formic acid. The final volume was reduced to <20 μL by evaporation in a vacuum centrifuge at ambient temperature. Sample volume was finally adjusted to ˜20 μL with 1% acetic acid.

### Recovery and Protein Stability Studies

Four peptide standard solutions of 50 μg/mL were prepared and processed under the exact conditions to which the plasma samples were subjected to test for the recovery of the entire procedure.

The stability of the protein to storage conditions was also checked. A fresh plasma sample of Atlantic salmon was separated into 4 aliquots. The first was frozen and stored at −20° C. The remaining three were subjected to 2, 4 and 6 freeze-thaw 24 hour cycles respectively and finally stored at −20° C. The four aliquots were analyzed as regular plasma samples, as described above.

### HPLC and MS/MS Analyses

### Part A

In the case of Rainbow trout and salmon, samples and standards were analyzed with a HPLC system (HP 1050 from Hewlett-Packard, Palo Alto Calif., USA) coupled on-line to a QqToF (QStar XL, Applied Biosystems, Foster City Calif., USA) tandem mass spectrometer equipped with an ESI ion source (TurbolonSpray). Reverse phase chromatographic separation was achieved on a 30×2 mm Gemini 5μ C18 110 Å column (Phenomenex, Torrance Calif., USA). All samples and standards were loaded into a 10 μL loop connected to a 10 port Valco (Houston, Tex., USA) switching valve. Table 1 shows the final solvent elution profile used for all the chromatographic separations.

TABLE 1
Optimized solvent elution profile and flow rates for the
chromatograhpic separation.

| Time | Flow rate | Solvent (%) | | |
|---|---|---|---|---|
| (min) | (μL/min) | A | B | C |
| 0.0-0.5 | 0.20 | 100 | 0 | 0 |
| 0.5-4.0 | 0.20 | 0 | 100 | 0 |
| 4.0-10.0 | 0.05 | 0 | 100 | 0 |
| 10.0-15.0 | 0.40 | 0 | 0 | 100 |
| 15.0-25.0 | 0.20 | 100 | 0 | 0 |

The parameters shown in this table were chosen to optimize the signals which were monitored during the MS/MS acquisition. Note that the flow rate was reduced from the 4^{th} minute to the 10^{th} minute, at which time the ‘signature peptide’ eluted from the chromatography column.

The product ion spectra were acquired in a ‘pseudo’ SRM mode. The mass spectrometer was programmed to alternatively monitor the product ions from the precursor ions at m/z 819.9 and 824.4 corresponding to the doubly charged species [M+2H]²⁺ of the ‘signature peptide’ and its deuterated isotopic homologue, respectively. This cycle was automatically repeated 750 times throughout the 25 minute chromatographic run.

The ToF detector was set to scan the product ions produced from m/z 800 to 1300. The tuning parameters of the mass spectrometer were optimized in terms of maximizing the signals produced by the m/z 819.9→(957.5−1028.57−1156.6) and the m/z 824.4→(960.5−1034.61−1165.7) transitions. The diagnostic product ions monitored correspond to the Y₉, Y₁₀ and Y₁₂ fragments of both the signature peptide and its labeled homologue.⁵⁵ These ions were additionally enhanced by setting the ion release delay (IRD) and the ion release width (IRW) parameters to 102.0 and 44.2 ms respectively, to increase the duty cycle of the ToF detector for these particular ions. The remaining tuning parameters used were: 4700 V for Ionization energy, 50 psi for nebulizer gas, 80 psi for auxiliary gas, 300° C. for probe temperature, 40 V for declustering potential (DP), 190 V for focusing potential, 15 V for DP2 and 36 V for the collision energy. The collision gas used was nitrogen at an arbitrary value of 5 set on the AnalystQS software. The first selecting quadrupole was operated at low resolution to further increase the product ion signal.

For quantification purposes, the extracted ion chromatograms of these diagnostic product ions were generated using a 2 Da mass range window around the selected product ions and manually integrated. The ratio between these two peak areas was taken as the dependant variable for the calibration curve. The same ratio was used for the digested plasma samples. These values were interpolated from the calibration curve, corrected by the corresponding dilution factors and finally converted to mg/mL of Vtg assuming a 1:1 molar ratio of signature peptide to Vtg molecule. The average molecular weights considered for Vtg were 181938 Da and 177153 Da for Rainbow trout and Atlantic salmon, respectively. These values were obtained from MALDI-MS experiments on the native intact Vtg proteins, reported in previous publications.^{10,11} Results were also expressed in μM units, considering an average molecular weight for the signature peptide of 1638.819 Da.

### Part B

### MALDI-QqToF-MS and MS/MS

In the case of cod and haddock, mass spectra of digests were acquired on a QSTAR-XL (Applied Biosystems, Framingham, Mass., USA) quadrupole-time-of-flight (QqToF) hybrid tandem mass spectrometer equipped with a UV nitrogen laser MALDI source (oMALDI 1, from Applied Biosystems, Framingham, Mass., USA). Ten microliters of the peptide extract belonging to one of the experimental fish and 10 μL of 2,5-dihydroxybenzoic acid (2,6-DHB) MALDI matrix were mixed together, spotted on the MALDI plate in 1 μL aliquots and allowed to evaporate at room temperature prior to mass spectral analysis. Upon optimization, typical acquisition parameters for the full scan positive ion mode were: Laser frequency 25 Hz, Laser power 30%, declustering potential (DP)-5V, focusing potential 255V and DP2 10V.

The product ion spectra were obtained by manually ‘ramping’ the collision energy from 30V to 120V during the acquisition until the spectra showed an adequate pattern of complete fragmentation, shown by evenly distributed peaks across the complete m/z range analyzed. Argon gas at an arbitrary value of 6 was used for the low energy collision induced dissociation (CID) MS/MS experiments. All data acquired was processed on the AnalystQS software.

‘De novo’ sequencing of these spectra was performed using the algorithm supplied by the Bioanalyst software, and manually inspected to verify the assignment of the characteristic peptide fragment ions. A database search for the peptide mass fingerprint and the product ion was carried out—peptide mass fingerprint search parameters were completed as follows: only 1 missed cleavage was allowed, carbamidomethyl cystein was chosen as the only variable modification to account for the alkylation reaction performed with iodoacetamide and the peptide mass tolerance was kept at 0.2 Da. No other restriction was applied in order to maximize discrimination.

### Signature Peptide Selection

In accordance with one embodiment of the present invention, the selected signature peptide used in the method relative to Rainbow trout and Atlantic salmon was one which has the following sequence:

Thr-Tyr-Phe-Ala-Gly-Ala-Ala-Ala-Asp-Val-Leu-Glu-Val-Gly-Val-Arg.

In this preferred embodiment, selection of the above ‘signature peptide’ was based on previous ‘de novo’ sequencing studies performed on Vtg tryptic peptides.^{10,11}, In the art, the Vtg from Rainbow trout has been completely sequenced. On the other hand, only a few peptides of the Atlantic salmon Vtg were found in a database search since only the 284 N terminus (entry Q800N6) and the 175 C terminus (entry Q8UWG2) amino acids of this species have been sequenced. It has been determined from the inventors' studies that some peptides were found common to both species, which thus provides a reduced list of candidate peptides^{10,11}, where the present invention is utilized to determine Vtg in two or more fish species at the same time.

In the preferred embodiments of the present invention, it is most desirable and efficient to choose a common peptide, common to two or more fish species, as a standard to enable use of the same standard calibration curve and permit one to analyze samples of both species in one batch. This feature can be compared to the ‘cross reactivity’ of immunological assays which allows homologous proteins sharing common epitopes to be detected using the same polyclonal and/or monoclonal antibodies.

In choosing a common signature peptide between two or more fish species, it is necessary to consider whether the signature peptide to be used in the present invention has an identical peptide sequence which could be found in nature in any other protein other than Vitellogenin. Although the genome of both species has not yet totally been elucidated, a BLAST search against all known protein and DNA databases with no taxonomic restriction was performed and provided no significant matches.

Again, in practicing the present invention, one should consider the amino acid composition of potential candidate sequences, which can be an influential factor in the final selection of the signature peptide to be used. Generally speaking, peptides containing methionine and cysteine are not favoured because of their propensity to form oxidized residues under different experimental conditions. Also, non-reactive, small hydrophobic residues are preferred over others due to their higher stability. Furthermore, tryptic peptide sequences starting with or having proline following the terminal lysine or arginine, i.e. X—X—R/K—P—X, being X any other amino acid, are not desirable because, when found at these positions, these amino acids are known to block the proteolytic action of trypsin.

It should be noted that the presence of post translational modification in the signature peptide location of the protein could also weaken the technique in two ways: it would increase the probability of peptide heterogeneity and increase enzymatic blockage during sample digestion. The selected ‘signature peptide’ lacked both glycosylation and phosphorylation consensus motifs, reducing the chances of these post translational modifications to occur.

In accordance with another embodiment, another preferred signature peptide has the following sequence Phe Phe Gly Gln Glu Ile Ala Asn Ile Asp Lys.

In accordance with another embodiment, another preferred signature peptide has the following sequence Glu Ile Val Leu Leu Gly Tyr Gly Thr Met Ile Ser Lys.

In accordance with another embodiment, another preferred signature peptide has the following sequence Tyr Glu Ser Phe Ala Val Ala Arg.

### Tuning and Optimization of Acquisition Parameters

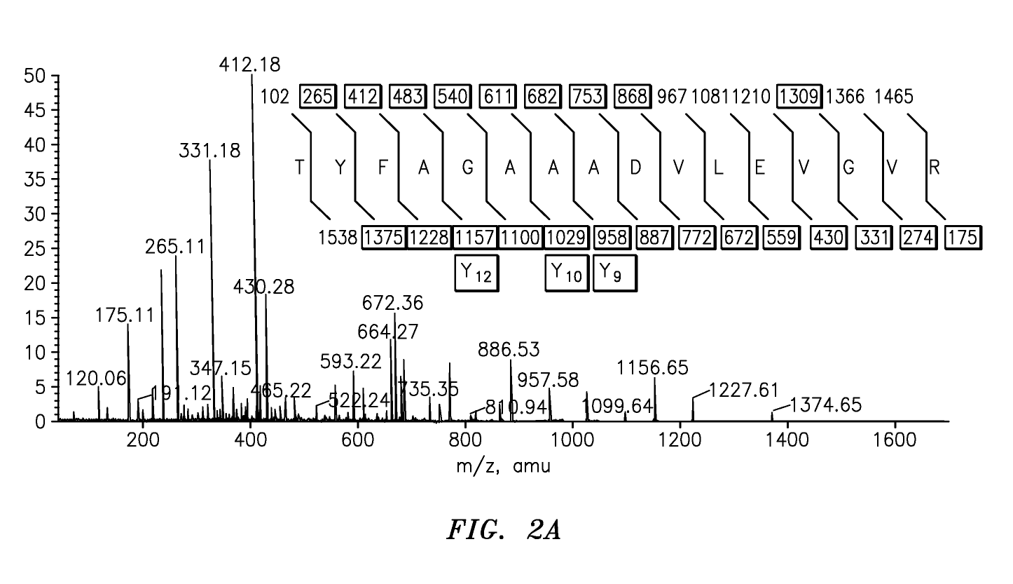

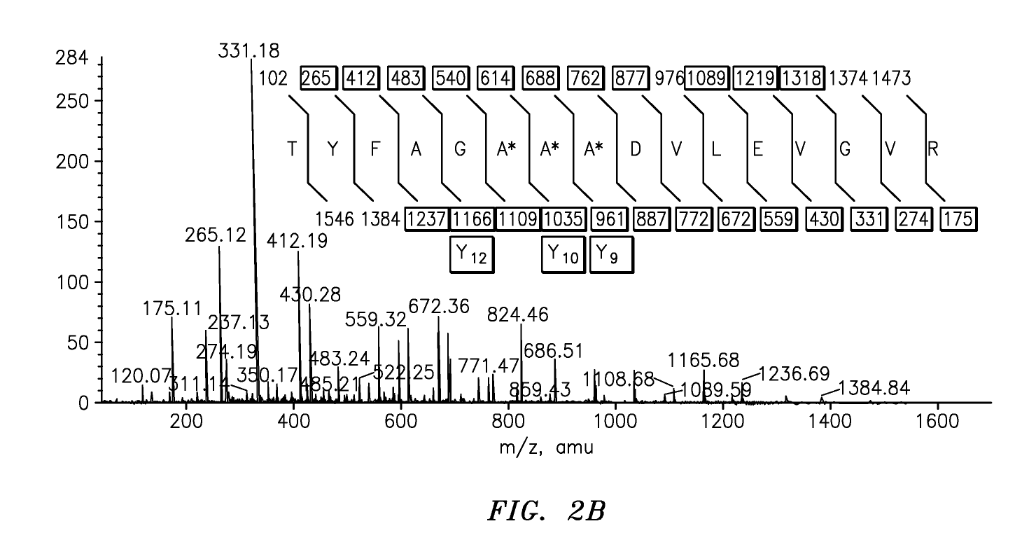

Referring again to FIG. 2, and by way of further explanation, this Figure shows the CID-MS/MS spectra obtained from the standard signature peptide and its deuterated homologue when infused directly into the mass spectrometer. The insets indicate the b- and y-type product ions expected from the classical peptide fragmentation routes.⁵⁵ Also, highlighted are the diagnostic Y₉, Y₁₀ and Y₁₂ product ions at m/z 957.5, 1028.57, 1156.6 and at m/z 960.5, 1034.61, 1165.7 which were monitored during the ‘pseudo’ SRM-MS/MS experiments. The decision to monitor these particular ions was based on a compromise between sensitivity and specificity. The fact that the product ions monitored had higher m/z value than that of the selected doubly charged precursor ions allowed for an increase in the specificity of the method. Under this criterion, any singly charged ion precursors with the same m/z as the signature peptides were neglected. However, this decision compromised the sensitivity of the method, given that some less specific product ions below m/z 400 would have rendered a much higher intensity signal as shown in FIG. 2.

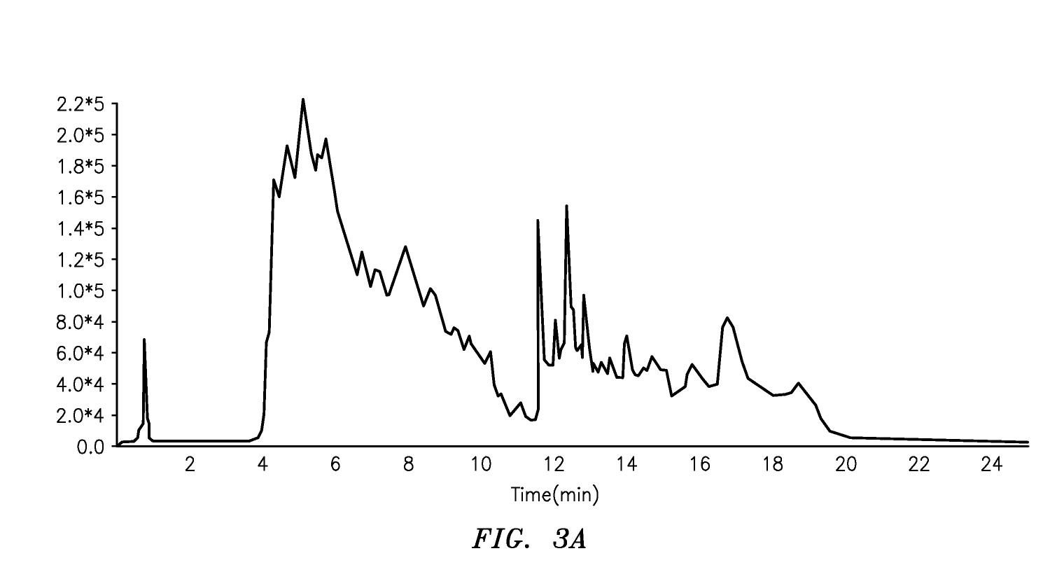

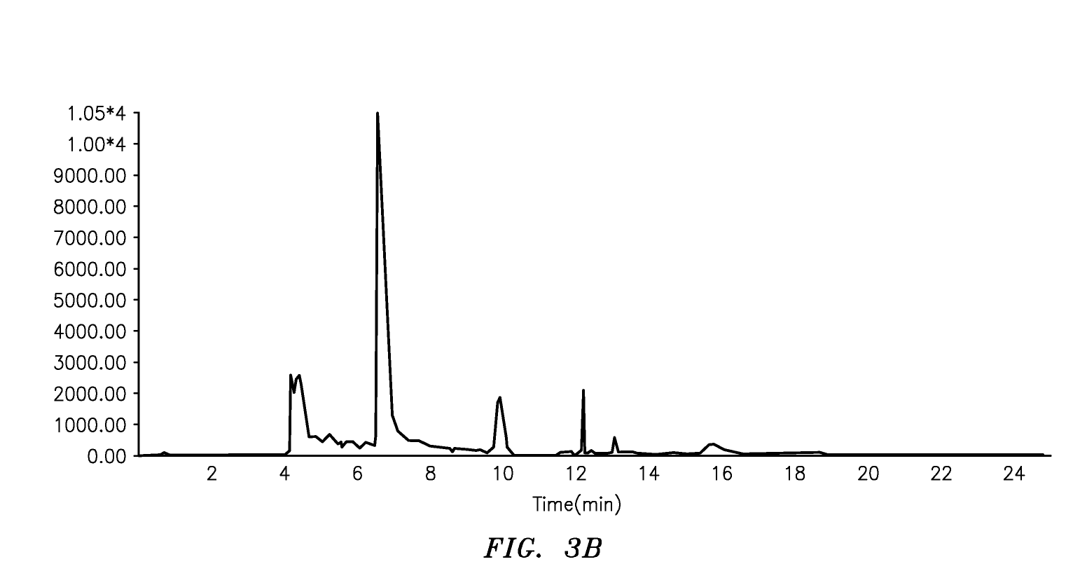

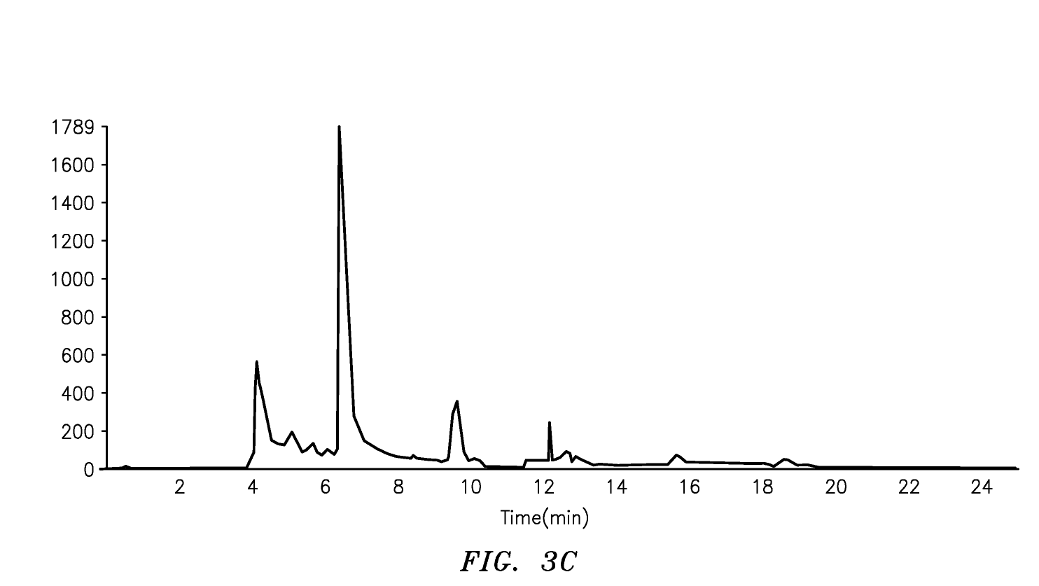

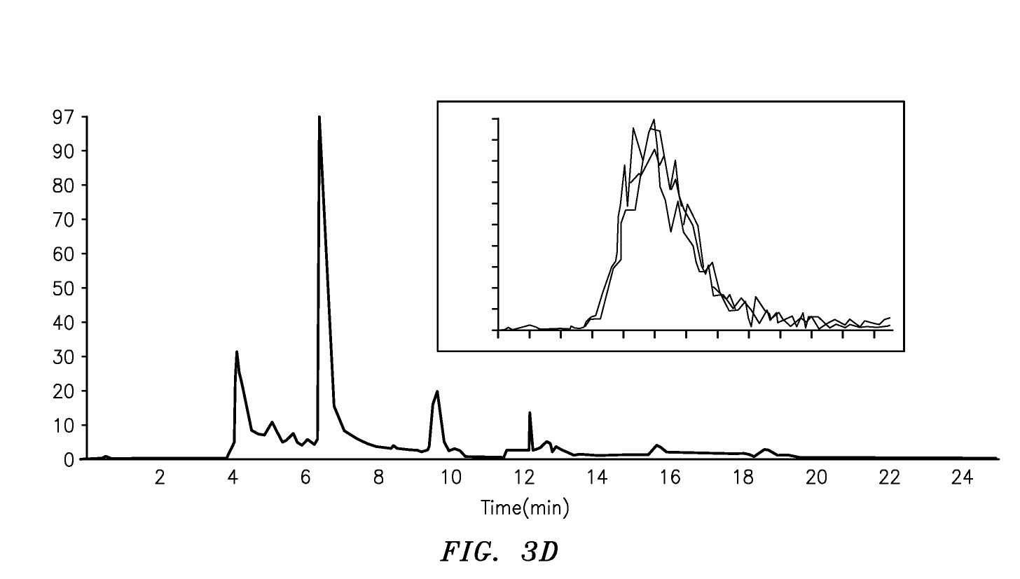

During the chromatographic separations of a complex plasma tryptic digest, the chances of encountering a peptide with a similar m/z value to that of the signature peptide, even at overlapping retention times, can not be ruled out. This situation is illustrated in FIG. 3. The chromatogram of a plasma tryptic digest was acquired in both MS and MS/MS modes and the corresponding extracted chromatograms for the precursor and product ions are shown. FIG. 3A shows the complexity of the protein digest illustrated by the conglomeration of peaks present in the total ion count (TIC) chromatogram acquired in full scan mode. This chromatogram was simplified when the [M+2H]²⁺ signature peptide at m/z 819.9 was extracted (see FIG. 3B). However, as expected for such a complex peptide mixture, other peaks at different elution times were also observed for that particular mass. The same sample was acquired in the ‘pseudo’ SRM scanning mode (see FIG. 3C). As expected, this chromatogram is very similar to that found in FIG. 3B, since both signals are generated or reconstructed from the same selected masses. FIG. 3D shows the added specificity given by tandem mass spectrometry. This figure shows the overlaid extracted ion chromatograms (EIC) corresponding to the Y₉, Y₁₀ and Y₁₂ diagnostic product ions at m/z 957.5, 1028.57 and 1156.6 obtained from FIG. 3C. The traces of these three chromatograms appear as superimposed (An expanded scale of them is shown in the inset of FIG. 3D). None of these showed any interfering peaks at other retention times, proving the enhanced specificity of this technique. Furthermore, these chromatograms show reduced baselines and increased signal to noise ratios compared to the TIC chromatograms obtained from the precursor ions.

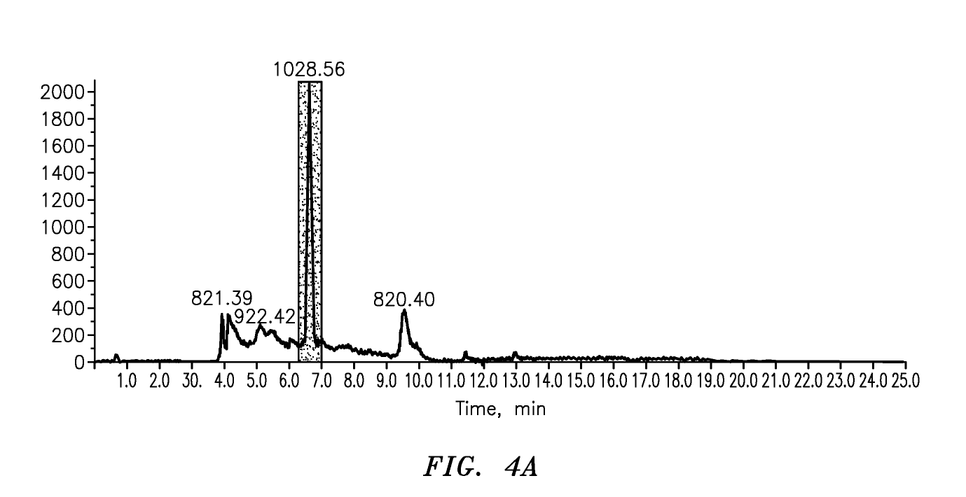

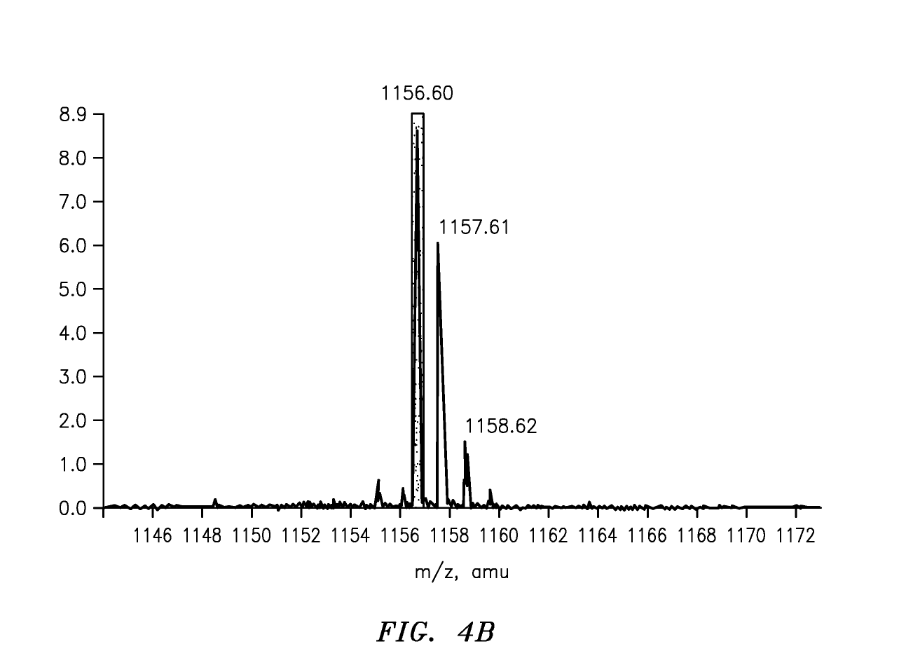

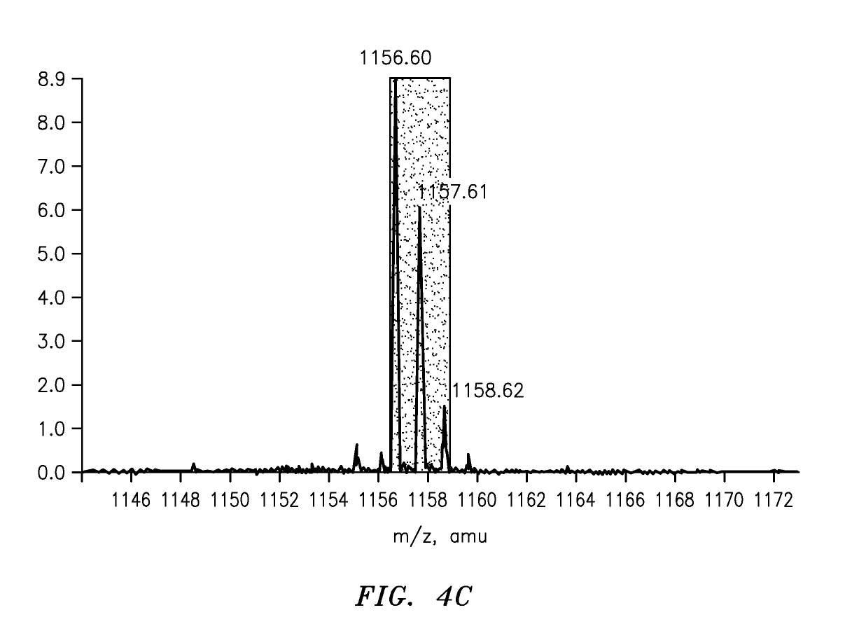

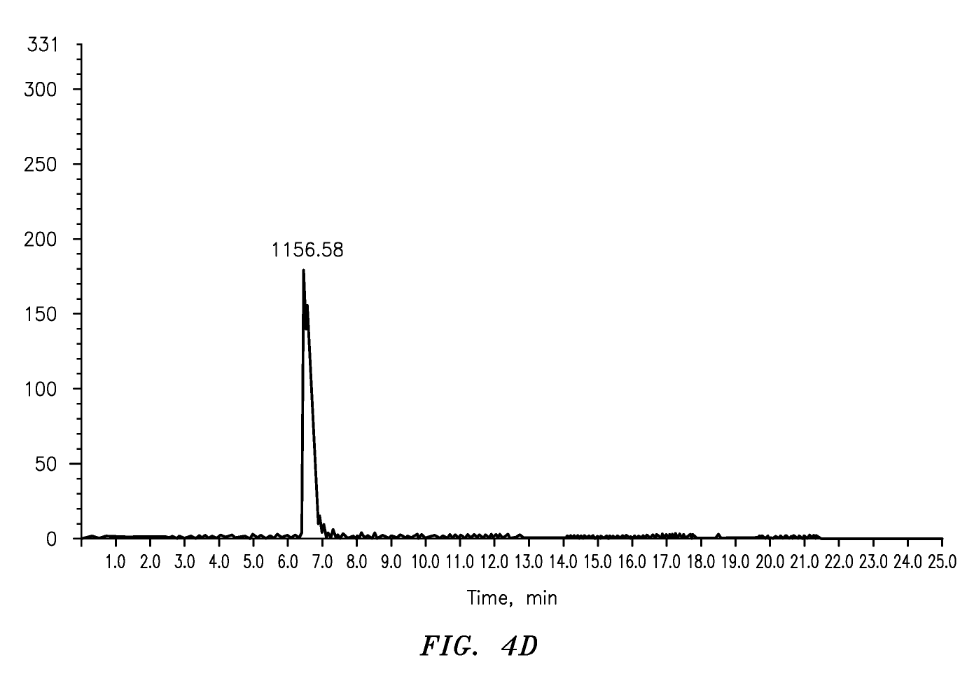

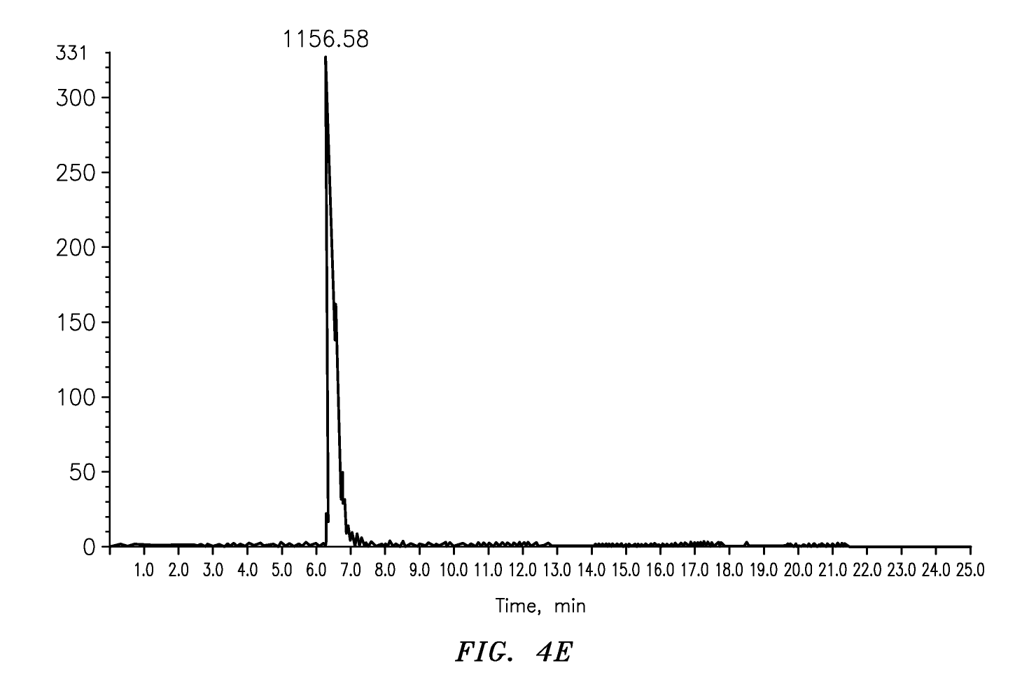

The extraction of ions from a TIC chromatogram using a TOF detector operated in pseudo-SRM mode is a post-acquisition process. To test whether the increased resolution of the QqTOF offers any advantage over the QqQ, the extracted chromatograms were generated using different mass ranges. FIG. 4A shows the TIC chromatogram acquired in the ‘pseudo’ SRM scanning mode of the precursor at m/z 819.9 from a plasma digestion sample. FIGS. 4B and 4C show the windows of 0.3 and 2.0 Da used around the Y₁₂ product ion at m/z 1156 to obtain the EICs shown in FIGS. 4D and 4E, respectively. The 0.3 Da window includes the monoisotopic peak exclusively while the 2.0 Da window is wide enough to include the complete isotopic cluster.

As shown in this particular case, there was no clear increase in specificity by using the higher resolution offered by the QqTOF. None of the extracted chromatograms (FIGS. 4D and 4E) showed a difference in their profile, i.e. no interfering peaks were observed in either chromatogram. However, sensitivity was compromised by half with the narrower extraction window (FIG. 4D), as showed by the less intense peak. Identical results were obtained from the extracted ion chromatograms of the Y₉ and Y₁₀ product ions respectively, at m/z 957.5 and 1028.57 (Data not shown). Moreover, the use of a wider window would avoid any inconvenience caused by a subtle modification in the ToF calibration parameters, where a slight change would be translated into increased peak integration errors during automated batched data acquisition analysis. Therefore, all subsequent extracted chromatograms were analyzed using a mass range window of 2 Da.

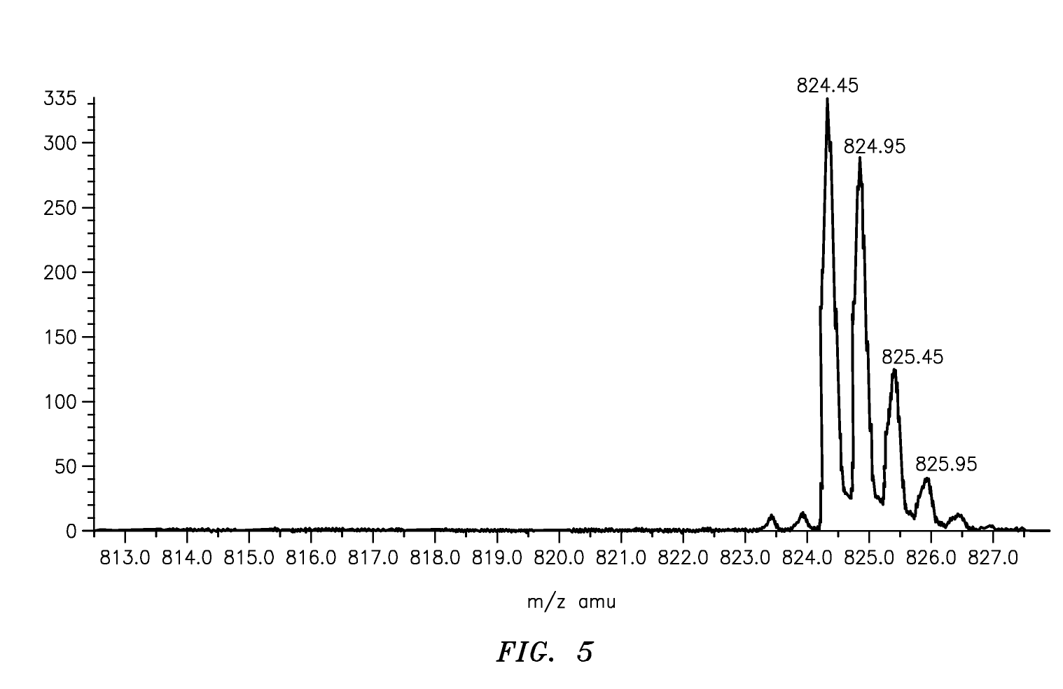

The internal standard was directly infused into the mass spectrometer and analyzed by TOF-MS to confirm that no residual L-Alanine had been incorporated instead of L-Alanine-3,3,3-d₃ during its synthesis. The absence of any peak at m/z 819.9 in the spectrum (FIG. 5) confirmed the labeled standard did not contain traces of the unlabeled standard. This requirement was critical, since all the samples analyzed in this study were spiked with the internal standard.

### Samples Analysis

Once all of the experimental parameters were adjusted, a calibration curve was constructed for each of the product ions monitored in the ‘pseudo’ SRM-MS/MS experiments. The linear regression parameters obtained for Y₉, Y₁₀ and Y₁₂ were 10.553, 10.389, 10.379 mL/μg for the slope, 0.0212, 0.0029, 0.0135 for the Y-intercept and 0.9988, 0.9966, 0.9983 for the R², respectively. The values obtained for these parameters again confirm the robustness of this method.

The limit of quantification (LOQ) for this method was calculated as the interpolated concentration produced by a baseline noise peak multiplied by a factor of ten. This translated into a value of 0.0008 ug/mL of signature peptide, equivalent to 0.009 mg/mL of Vtg.

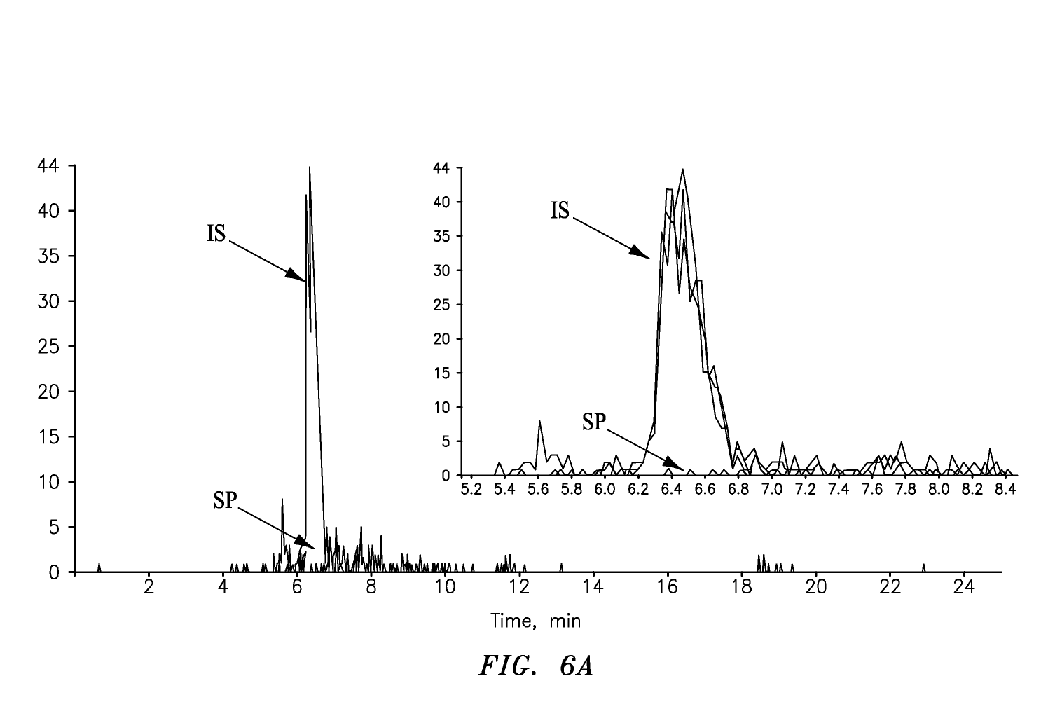

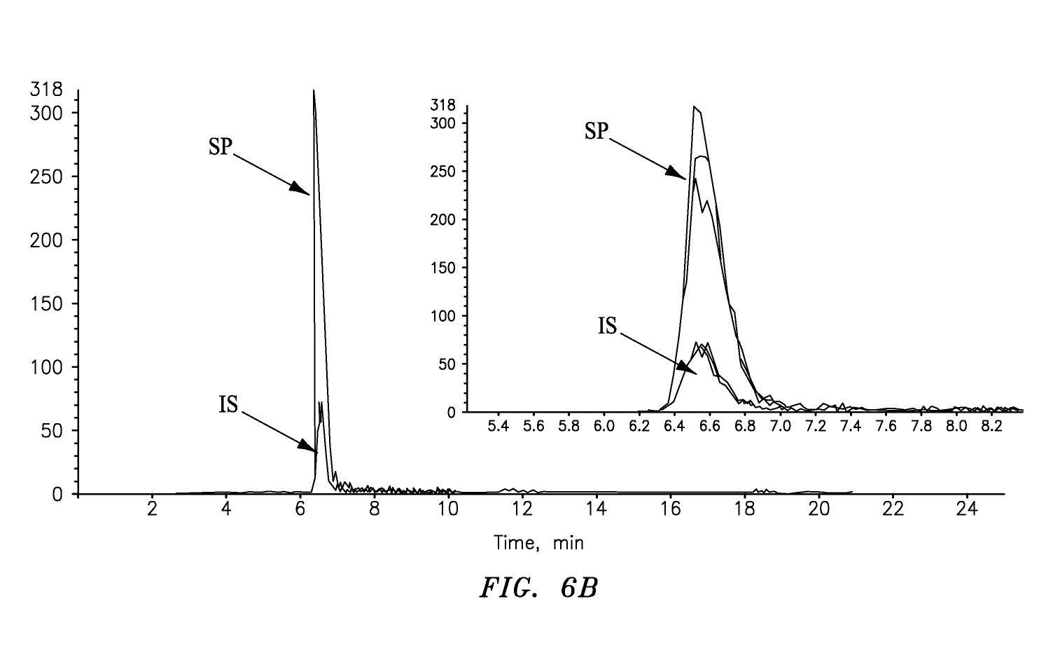

The digested plasma samples were then run under the same conditions as the standards. FIG. 6 shows of a Rainbow trout sample prior to (FIG. 6A) and after β-estradiol induction (FIG. 6B). These figures show the overlaid extracted ion chromatograms of the Y₉, Y₁₀ and Y₁₂ product ions of both signature peptide and labeled internal standard at m/z 957.5, 1028.57, 1156.6 and m/z 960.5, 1034.61, 1165.7, respectively. The traces obtained from the ‘signature peptide’ before β-estradiol induction were hardly distinguishable from the baseline. The traces corresponding to the internal standard formed a clear peak in both samples. The inset shows the expanded scale of these traces, confirming the absence of Vtg before treatment (FIG. 6A).

The signature peptide and equivalent Vtg plasma concentrations from all the samples analyzed can be seen in Table 2, as follows:

TABLE 2
Rainbow trout (RT) and Atlantic salmon (AS) signature peptide (SP) and
Vtg concentration results obtained from both control (C) and experimental (E)
fish.

| | | Before β-estradiol | | | After β-estradiol | | | |
|---|---|---|---|---|---|---|---|---|
| | | SP Conc | Vtg Conc | | SP Conc | Vtg Conc | | |
| Sample | Type | μg/mL | μg/mL | μM | μg/mL | μg/mL | μM | t-test P |
| RT | C | 0.000 | 0.000 | 0.000 | 0.096 | 1.061 | 0.058 | 0.11 |
| RT | C | 0.041 | 0.453 | 0.025 | 0.085 | 0.943 | 0.052 | |
| RT | E | 0.000 | 0.000 | 0.000 | 0.576 | 6.389 | 0.351 | <<0.001 |
| RT | E | 0.000 | 0.000 | 0.000 | 0.799 | 8.867 | 0.487 | |
| RT | E | 0.068 | 0.756 | 0.042 | 0.562 | 6.239 | 0.343 | |
| RT | E | 0.000 | 0.000 | 0.000 | 0.456 | 5.059 | 0.278 | |
| RT | E | 0.000 | 0.000 | 0.000 | 0.615 | 6.830 | 0.375 | |
| RT | E | 0.029 | 0.327 | 0.018 | 0.771 | 8.562 | 0.471 | |
| RT | E | 0.000 | 0.000 | 0.000 | 0.197 | 2.182 | 0.120 | |
| RT | E | 0.000 | 0.000 | 0.000 | 0.368 | 4.091 | 0.225 | |
| RT | E | 0.000 | 0.000 | 0.000 | 0.185 | 2.057 | 0.113 | |
| RT | E | 0.000 | 0.000 | 0.000 | 0.564 | 6.264 | 0.344 | |
| RT | E | 0.025 | 0.272 | 0.015 | 0.754 | 8.370 | 0.460 | |
| AS | C | 0.008 | 0.088 | 0.005 | 0.054 | 0.598 | 0.033 | 0.23 |
| AS | C | 0.290 | 3.217 | 0.177 | 0.209 | 2.325 | 0.128 | |
| AS | C | 0.000 | 0.000 | 0.000 | 0.063 | 0.701 | 0.039 | |
| AS | C | 0.000 | 0.000 | 0.000 | 0.103 | 1.145 | 0.063 | |
| AS | E | 0.000 | 0.000 | 0.000 | 0.628 | 6.967 | 0.383 | <<0.001 |
| AS | E | 0.223 | 2.476 | 0.136 | 0.969 | 10.758 | 0.591 | |
| AS | E | 0.000 | 0.000 | 0.000 | 0.439 | 4.876 | 0.268 | |
| AS | E | 0.000 | 0.000 | 0.000 | 0.653 | 7.254 | 0.399 | |
| AS | E | 0.008 | 0.086 | 0.005 | 0.540 | 5.997 | 0.330 | |
| AS | E | 0.000 | 0.000 | 0.000 | N/A | N/A | N/A | |

The identities of the samples analyzed were confirmed by the presence of all three product-ions (Y₉, Y₁₀ and Y₁₂) in the ‘pseudo’ SRM analyses at the corresponding elution time of the signature peptide. For quantification purposes, the concentrations obtained from each of the diagnostic product ions were averaged together. The coefficient of variation was typically below 10% among the three concentrations obtained for each sample. Concentration values of zero analytically mean less than the LOQ. As expected, most of the juvenile Rainbow trout had non-detectable plasma levels of Vtg before β-estradiol induction, except for a couple of larger female trout which were probably at stages of early sexual maturation. As for the Atlantic salmon, two samples were confirmed as females, after the animals were killed and their ovaries found filled with eggs. This would explain the high levels of Vtg found in some of the fish before β-estradiol induction, characteristic of the vitellogenic reproductive phase.

A t-test for paired samples was applied to each one of the groups (control Rainbow Trout, experimental Rainbow trout, control Atlantic salmon and experimental Atlantic salmon). As expected, a statistically significant (P<<0.001) increase in Vtg plasma concentration was established for all experimental fish, whereas no significant increase was observed for the control fish.

The results from the recovery tests are depicted in following Table 3.

TABLE 3
Statistical analysis done on the recovery studies. The peptide
concentration for each recovery sample was calculated as the average
concentration obtained from each of the Y9, Y10 and Y12 calibration
curves. An average of these, the standard deviation (SD) and the
coefficient of variation (CV) percent were calculated. Recovery % was
based on 0.1 ug/mL for 100% recovery.

| | Peptide Conc. | Average Conc. | SD | | Recovery |
|---|---|---|---|---|---|
| Sample | μg/mL | μg/mL | μg/mL | CV % | % |
| R1 | 0.070 | 0.067 | 0.002 | 3.4 | 67 |
| R2 | 0.069 | | | | |
| R3 | 0.067 | | | | |
| R4 | 0.064 | | | | |

Reproducibility of this methodology was within acceptable values having a coefficient of variation of 3.4%. However, recovery results averaging 67% were not as good as expected. Nevertheless, because of the systematical and reproducible loss of peptide under the described experimental conditions, these recovery results could be incorporated into a correction factor to account for peptide loss during sample handling and experimental procedure.

A low recovery factor may be due to the chemical transformation of the peptide. The enzymatic digestion occurs under mild reacting conditions (37° C. and pH 7.4), however the previous denaturing step takes place at 60° C. in 8M urea. Carbamylation of terminal NH₂ groups in proteins could occur under these conditions as described in literature.^{56,57,58} To check for this and any other possible chemical modifications of the standard peptide, an Information Dependant Acquisition (IDA) survey scan was performed on one of the recovery sample tubes. More than 15 product ion spectra, excluding that corresponding to the signature peptide, were obtained from all the additional minor peaks present in the chromatogram to investigate whether any of these were derived from the signature peptide (Data not shown). A meticulous analysis of all these spectra showed that none of them contained the carbamoylated sequence tags characteristic of the signature peptide.

The inventors have also considered the issue of whether the method of the present invention, which requires complete or substantially complete digestion of the plasma samples, would be affected by the steps of the present invention. It has been established that by following the parameters of the present invention, substantially complete digestion, at least in the region of the protein, can be obtained. This was verified by the following: a series of hypothetical candidate sequences corresponding to the product of enzymatic missed cleavages of the signature peptide were proposed. These can be observed in the following Table 4:

TABLE 4

Calculated m/z for the single and double charged ions
of signature peptide(*) and candidate peptides for the
incomplete digestion of Rainbow trout Vtg. Cleavage
sites are shown with a dash sign (-).

| | | |
|---|---|---|
| | [M + H]⁺ | [M + 2H]²⁺ |
| Peptide sequence | m/z | m/z |

| | | |
|---|---|---|
| TYFAGAAADVLEVGR* | 1638.84 | 819.92 |
| AR-TYFAGAAADVLEVGR | 1865.98 | 933.49 |
| TYFAGAAADVLEVGR-TEGIQEALLK | 2721.44 | 1361.22 |
| AR-TYFAGAAADVLEVGR-TEGIQEALLK | 2948.58 | 1474.79 |
| TVVAK-AR-TYFAGAAADVLEVGR | 2364.30 | 1182.65 |
| TYFAGAAADVLEVGR-TEGIQEALLK-PPAPENADR | 3781.97 | 1891.49 |

Subsequently, digested plasma samples were injected into the HPLC-QqToF-MS/MS and processed in the ‘pseudo’ SRM mode, except that a different precursor ion from Table 4 was selected for each run. The TIC chromatograms obtained when each of these precursors were selected revealed some suspicious peaks, however the product ion spectra obtained from them showed no evidence of any of the fragment ions expected from the candidate peptides. Thus, digestion was complete, at least on this region of the protein.

The present invention has also considered whether conservation conditions of samples prior to analysis could affect final results. This is an important factor issue since blood sampling is usually performed in fieldwork, and infrequently under laboratory conditions, which could lead to a considerable time lag period between sampling and laboratory processing. This lag time increases the possibility of Vtg breakdown as this protein is known to be highly susceptible to degradation.⁵⁹ To determine this issue, conservation conditions were simulated by subjecting the same sample to increasing number of freeze-thaw cycles prior to analysis. The results of this study are portrayed in the following Table 5:

TABLE 5
Effect of freeze-thaw cycles on the final results obtained from an
Atlantic salmon plasma sample.

| Freeze-thaw | Peptide conc. | Average conc. | SD | |
|---|---|---|---|---|
| cycles | (μg/mL) | (μg/mL) | (μg/mL) | CV % |
| 1 | 0.431 | 0.451 | 0.019 | 4.17 |
| 3 | 0.449 | | | |
| 5 | 0.449 | | | |
| 7 | 0.477 | | | |

The conditions assayed in this test clearly do not affect the results obtained, and thus show one of the benefits of the method of the present invention. In addition, the reproducibility of the concentration values is consistent with that obtained from the recovery test.

From the above description, it will be seen that the present invention presents a simple and straightforward HPLC-MS/MS technique for the quantification of Vtg plasma levels in a plurality of fish species, such as Rainbow trout and Atlantic salmon. The use of a QqToF operated in the ‘pseudo’ SRM mode allows monitoring of three diagnostic product ions, enhancing specificity without compromising sensitivity. Further, the specificity was not increased by reducing the mass window of the extracted ion chromatograms from 2 Da to 0.3 Da. Moreover, sensitivity was reduced by a factor of approximately 2.

### Atlantic Cod/Haddock

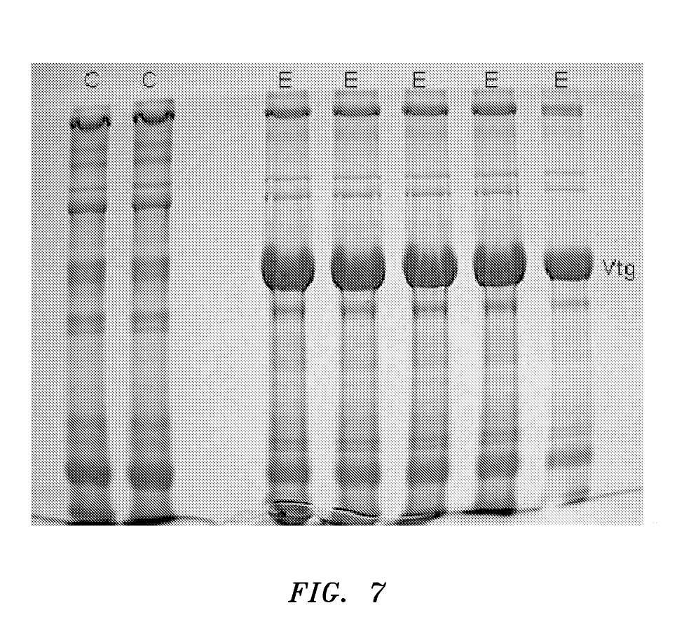

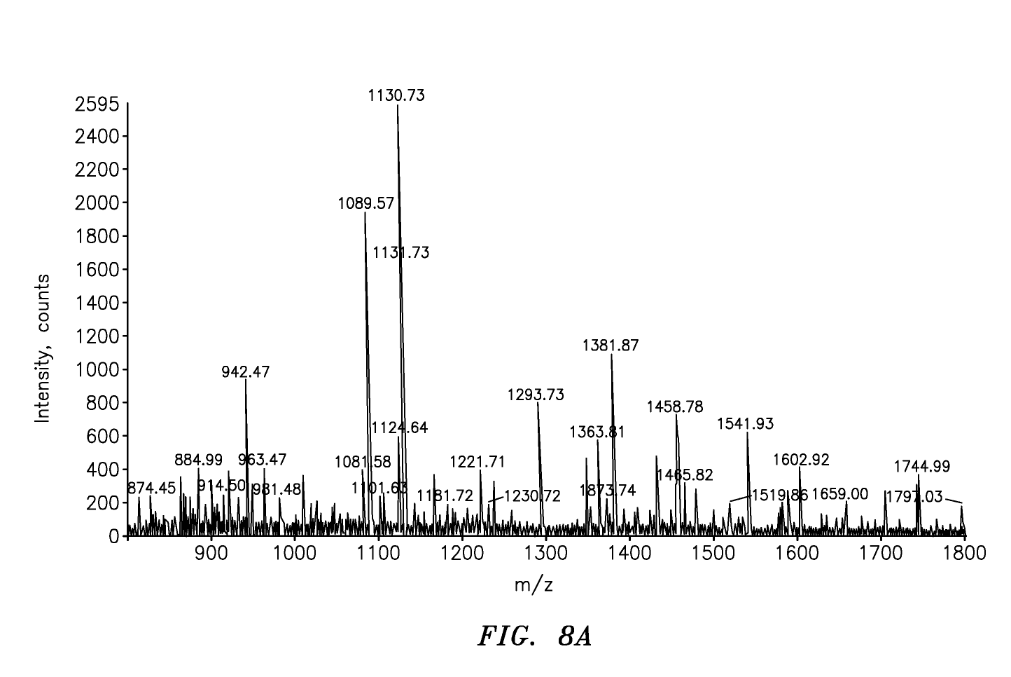

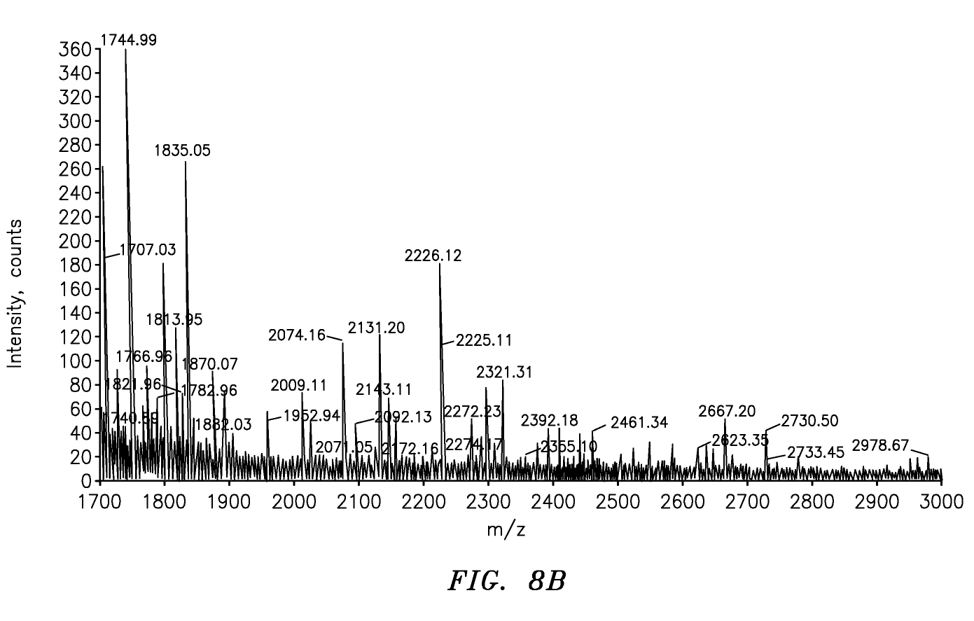

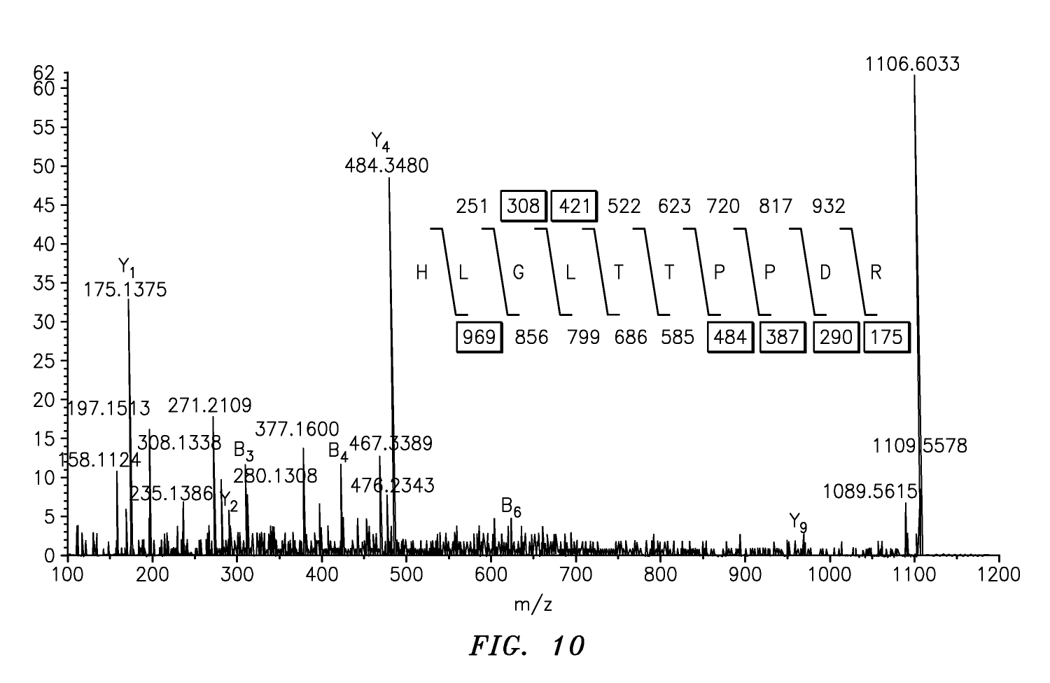

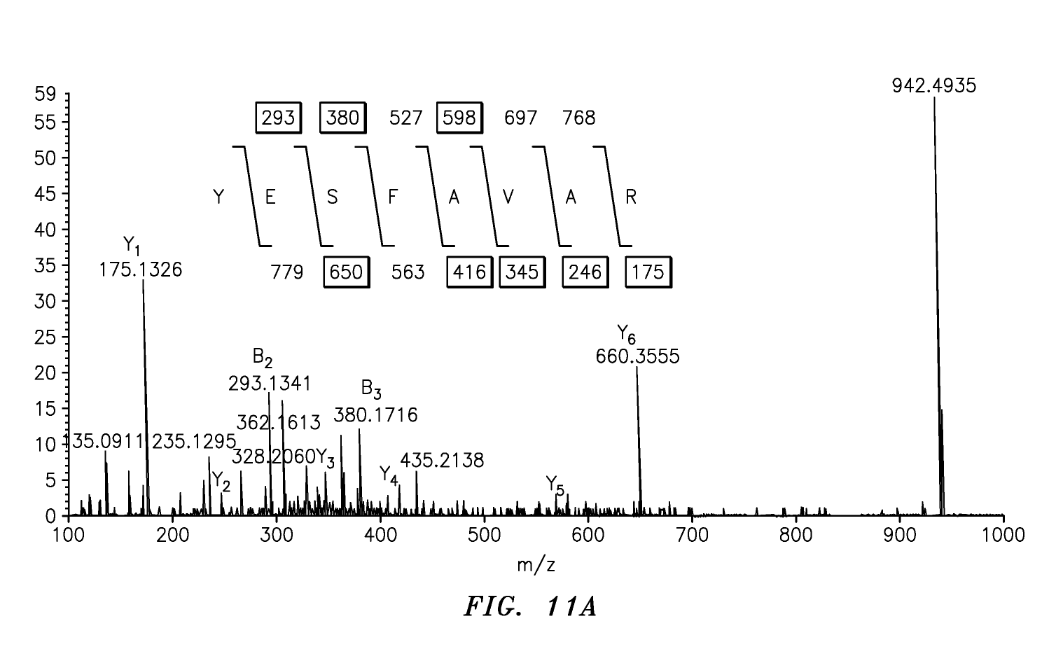

Referring now to FIGS. 7 to 11, the efficiency of Vtg induction with β-estradiol was assessed by SDS-PAGE as seen in FIG. 7. The strong induced band which appeared in the experimental fish was excised and subjected to ‘in-gel’ trypsin digestion. The peptides extracted from the digestion were subjected to MALDI-MS (FIG. 8).

Although no sequence for Atlantic cod Vtg is currently available, a comparison of the peptide ions was made based on those obtained in the enzymatic digestion with those generated ‘in-silico’ in protein databases, since Vtg sequences from other species have already been reported. For this purpose, the [M+H]⁺ ions present in the MALDI-MS spectra were used for protein identification using the Mascot Peptide Mass Fingerprint database search engine.

The two known different forms Haddock Vtg (VtgA and Vtg B) gave statistically significant hits with a search query (entries Q98T86 and Q98T87 on the TrEMBL database). Atlantic Cod and Haddock are two ground fish species which are evolutionarily related to one another. Both species belong to the family Gadidae, subfamily Gadinae (see reference⁶⁶). As described in literature, Haddock, in common with other species, co-expresses more than one form of Vtg⁶⁴. These forms have been shown to have different biological roles after being incorporated in the oocyte.

Analysis of the matching peptides between both species showed that some of the Atlantic Cod Vtg peptides matched those of Vtg A, others of Vtg B while some were common to both Vtg A and B, as shown in Table 6.

TABLE 6

| | | | | |
|---|---|---|---|---|
| [M + H]+ | | | | |
| m/ | Vtg A | Vtg B | Vtg A + B | Sequence |

| | | | | |
|---|---|---|---|---|
| 813.50 | | x | | IKHFIR² |
| 942.47 | x | | | YESFAVAR¹ |
| 948.46 | | x | | VAWGIDCK² |
| 963.46 | x | | | DLNNCQEK¹ |
| 1009.53 | x | x | x | DLGLAYTEK¹ and DIGLAYTEK² |
| 1081.57 | | x | | LPQAPVDADR² |
| 1089.56 | x | x | x | LCADGILLSK¹ and INLNAAFAKK² |
| 1101.63 | x | x | x | TEGLQEALLK¹ and TEGIQEALLK² |
| 1106.59 | | x | | CFSVEPVLR² |
| 1124.62 | x | | | VHVDAILAMR¹ |
| 1130.72 | x | x | x | FLELIQVLR¹ and FVELIQLLR² |
| 1166.61 | x | | | LESEDASFIR¹ |
| 1221.71 | | x | | MAAALVLFETR² |
| 1293.73 | | x | | FTWDKLPTSAK² |
| 1349.79 | x | | | IVKDLGLAYTEK¹ |
| 1363.81 | | x | | ITAATVETFAIAR² |
| 1372.70 | x | | | DNIEQNWINVK¹ |
| 1458.78 | | x | | FFGQEIGFASIDK² |
| 1465.82 | x | | | FLGQEIAFANLDK¹ |
| 1479.84 | x | | | TPIAPVNAQYLHR¹ |
| 1743.13 | x | x | x | IVPIIPAEILEPLIGR¹ and LTLALTSDKTLNIVLK² |
| 1796.01 | x | | | VYSPEGISTTVLNIFR¹ |
| 1834.05 | | x | | VQLILANLVEENHWR² |
| 2008.11 | x | | | ASATPALPQNFLWTHLLK¹ |
| 2020.06 | | x | | VHEDAPLKFVELIQLLR² |
| 2073.16 | | x | | AIIDQLIQVATGPSVATYGR² |
| 2225.11 | | x | | LASNSVSYAQSWVIPAESCR² |
| 2294.29 | x | | | LESEDASFIRNTPLYQLIGK¹ |
| 2320.29 | | x | | GSLQYEFATELLQTPIQLLR² |
| 2665.17 | x | | | DAVSYAHSWVIPAENCQDASECR¹ |

The latter are of particular interest since the ions at m/z 1009.53 and 1101.63 corresponded to pairs of peptides which were not identical, but contained leucine-isoleucine substitutions. The ion at m/z 1130.72 matched a pair of peptides in which two of their amino acids have switched positions. Alternatively, the ions at m/z 1089.56 and 1743.13 were assigned to pairs of isobaric peptides with absolutely no sequence homology between them.

Product ion spectra were acquired by MALDI-QqToF MS/MS to confirm the identity of both matching and non-matching peptide ions. The results of these analyses are shown in Table 7.

TABLE 7

| | | | |
|---|---|---|---|
| Precursor | | | |
| m/z | Proposed sequence | Comment | Haddock Vtg sequence |

| | | | |
|---|---|---|---|
| 942.49 | YESFAVAR | A | |
| 1009.53 | DLGLAYTEK | A + B | |
| 1081.56 | QPLMNDAHR | D | LPQAPVDADR |
| 1089.54 | FDDVEPVLR | D | CFSVEPVLR |
| 1106.58 | HLGLTTPPDR | E | |
| 1124.62 | VHVDALLAMR | A | |
| 1130.71 | FLEVLQLLR | C + D | FLELIQVLR and FVELIQLLR |
| 1166.60 | LESEPLLPLR | C | LESEDASFIR |
| 1221.67 | ACVALVLFETR | D | MAAALVLFETR |
| 1237.70 | RPGVVWDPVGR | E | |
| 1293.64 | WDTFQMQLPK | D | FTWDKLPTSAK |
| 1363.75 | SSPPGMETFALAR | D | ITAATVETFAIAR |
| 1381.81 | VPLPQFLPLAMR | E | |
| 1433.75 | ALAAGVDLTEGNFR | E | |
| 1458.74 | FFGAWVAFAEADK | D | FFGQEIGFASIDK |
| 1465.82 | FLGGPCLAFANLDK | C | FLGQEIAFANLDK |
| 1479.81 | EPQAAPVNTFLHR | C | TPIAPVNAQYLHR |
| 1519.76 | (805.41)EWSHR | E | |
| 1540.90 | GVAAGFLPLAASLTPR | E | |
| 1588.90 | (789.3369)VLELGLR | E | |
| 1602.90 | TTLGSFNVDALLPVR | E | |
| 1704.95 | HSAATVLESWTFTVR | E | |
| 1743.13 | (833.51)LLEPLLGR | C | IVPIIPAEILEPLIGR |
| 1796.01 | (1133.59)LNLFR | C | VYSPEGISTTVLNIFR |
| 1834.97 | HPHQRPLDLENHWR | D | VQLILANLVEENHWR |
| 2073.99 | (975.5507)NPYQVAGGHR | E | |
| 2131.02 | NAVGSGYNHTFEHATVATVR | E | |
| 2226.00 | (1410.56)LPASSGGAR | E | |

A: Perfect match with Haddock Vtg A sequence
B: Perfect match with Haddock Vtg B sequence
C: Partial match with Haddock Vtg A sequence
D: Partial match with Haddock Vtg B sequence
E: No match with Haddock Vtg

As will be seen from both Table 6 and Table 7, the signature peptide for both Atlantic cod and haddock is disclosed, which signature peptide is Asp Leu Gly Leu Ala Tyr Thr Glu Lys, with m/z at 1009.53. This represents the preferred signature peptide for these two species of fish.

Computer programs relating to the sequencing of peptides have made, compared to earlier intensive time consuming procedures, the task faster but care has to be taken to make sure that the accuracy is that required for this purpose. When such programs are used, a list of candidate peptides can be shown in score order. Such score depends, among other parameters, on the number of matching fragment ions and their respective mass error. Nevertheless, it is highly desirable that each candidate sequence be manually checked to avoid errors in peak assignment, especially with regard to the correct identification of the monoisotopic peak. Therefore, the candidate sequences shown in Table 7 correspond to those, which according to the desired criteria best matched the MS/MS spectra, but did not necessarily match with the highest score.

It has been found that not all the ions located in the peptide mass fingerprint were amenable to MS/MS analysis. Some spectra did not yield sufficient diagnostic peaks to permit the full sequencing of the peptides. Thus, there would be some missing precursor ions in Table 7—nevertheless, partial sequences could be obtained. In these cases a mass value between brackets is shown to account for the amino acid sequence gaps.

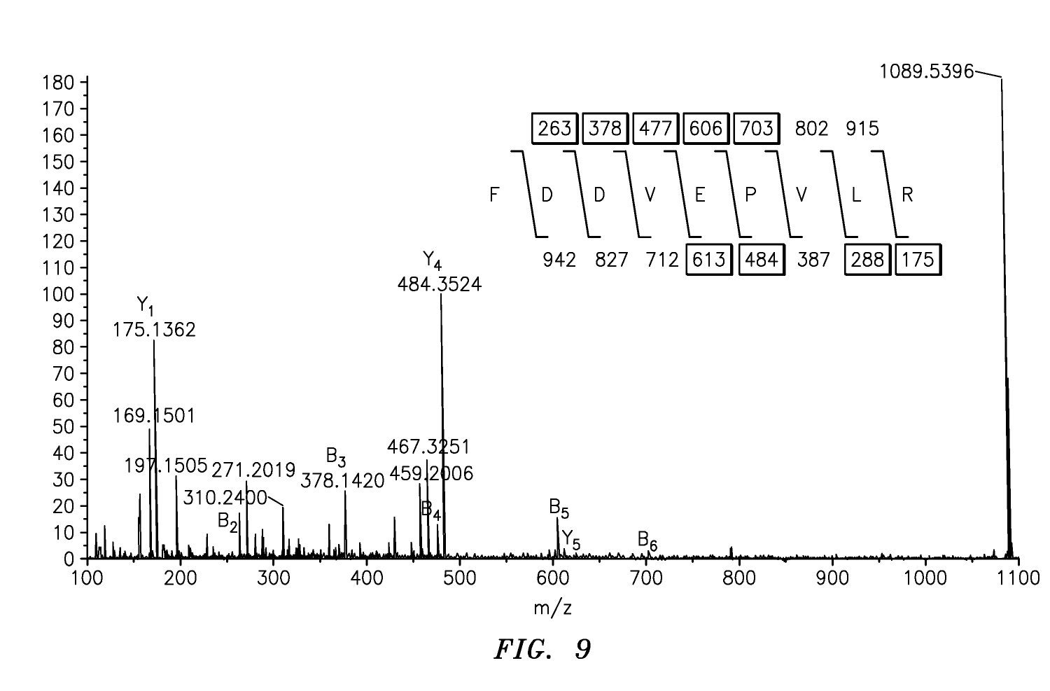

Examination of Table 7 reveals that the product ion spectrum of the precursor ion at m/z 1089.54 (see FIG. 9) has a sequence similar to that found in Vtg B of Haddock, yet the peptide mass fingerprint had erroneously assigned this ion to other Haddock peptides. There is evidence to support this new sequence (FDDVEPVLR), since the mass of this peptide differs from the theoretical mass of the corresponding Haddock peptide (CFSVEPVLR, Mr 1105.56). Interestingly, the product ion spectrum of the ion at m/z 1106.58 (see FIG. 4) showed no apparent similarity to the peptide assigned to it on the peptide mass fingerprint.

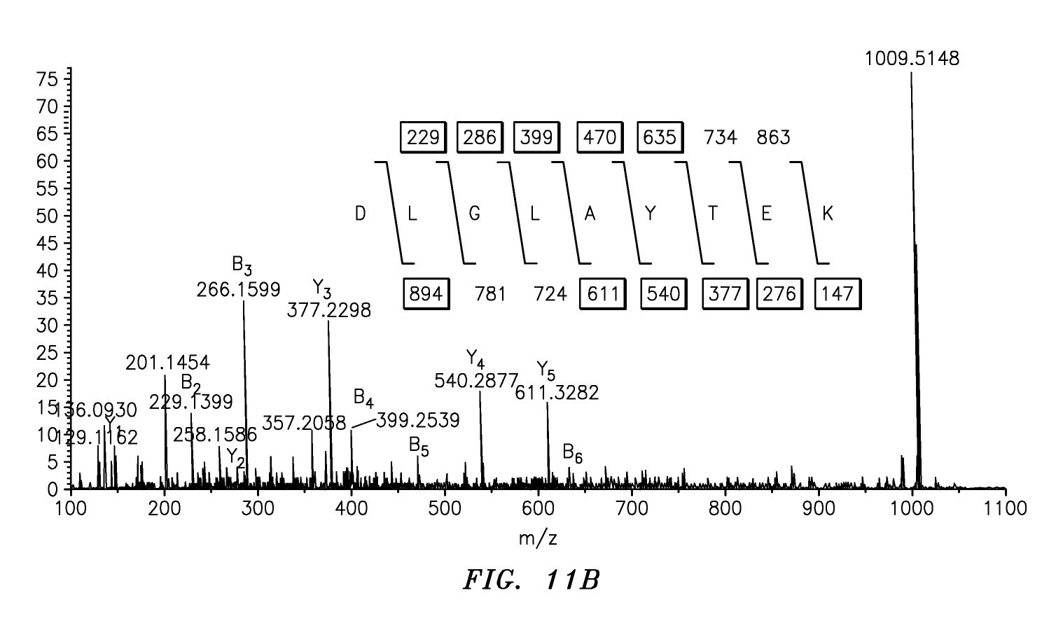

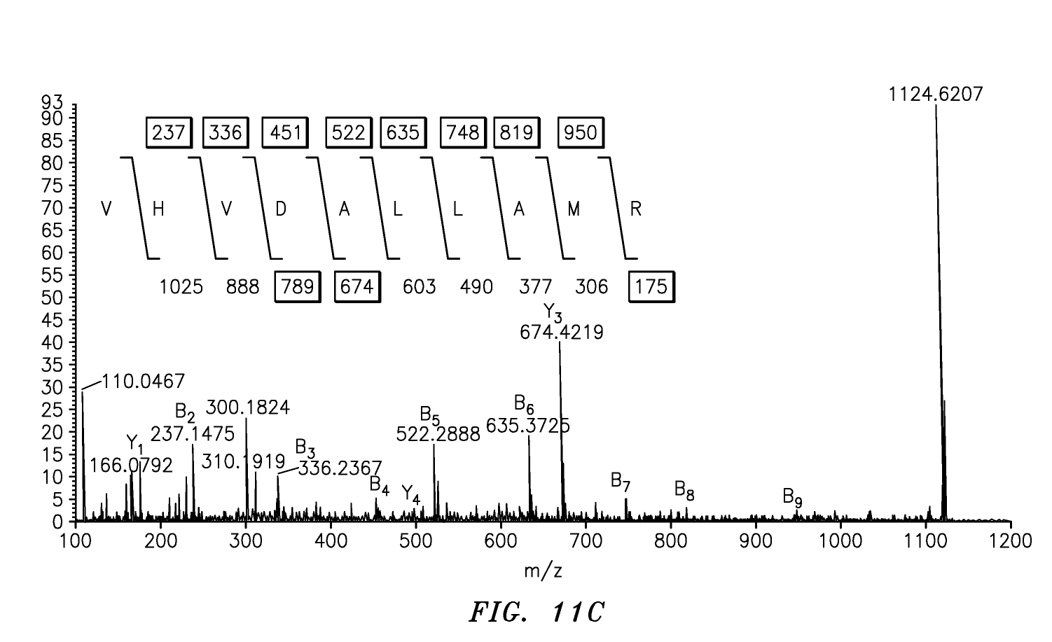

The product ions spectra of ions at m/z 942.49, 1009.53 and 1124.62, shown in FIGS. 11A, 11B and 11C had a perfect match with their corresponding Haddock tryptic peptides. Note that leucine and isoleucine cannot be distinguished from each other for sequencing purposes and are marked with the letter L by the sequencing software to indicate both amino acids.

The product ion spectrum of the selected precursor ion at m/z 1743.13, which had been assigned to two different peptides on Table 6, was partially sequenced to match the Vtg A of Haddock Vtg. The remaining ions showed either partial or no sequence homology with the Haddock Vtg sequence.

It is important to note that there is a series of ions obtained in the conventional MALDI-MS at m/z 826.47, 884.98, 920.43, 1123.61, 1952.94, 2142.12, 2391.19, 2408.18, 2440.36, 2460.33, 2585.56, 2637.35, 2729.49, 3387.66, 3435.72 and 3450.78, which did not match any of the predicted Haddock Vtg tryptic peptides with the chosen database search parameters. These ions were designated as diagnostic to Atlantic cod, and are presently being explored.

The appearance of Atlantic cod tryptic peptides that match both Vtg sequences found in Haddock is evidence to propose that Atlantic cod might also co-express at least two forms of Vtg. This is further supported by the finding of ions at m/z 1458.74 and 1465.82. These two overlapping variant peptides belonging to Haddock Vtg B (FFGQEIGFASIDK) and A (FLGQEIAFANLDK) have been simultaneously extracted from the trypsin digestion of the Atlantic cod Vtg band in the SDS-PAGE.

From the above, it will be seen that the characterization of Atlantic cod (Gadus morhua) vitellogenin, resulted in a protein whose amino acid sequence is yet unknown. Vitellogenin synthesis was induced ‘in vivo’ and subjected to trypsin digestion for analysis by matrix-assisted laser desorption/ionization-Quadrupole-Time-of-flight tandem mass spectrometry.

In addition, the above illustrates that a peptide mass fingerprint can be obtained and ‘de novo’ sequencing of the most abundant tryptic peptides can also be achieved by low energy collision induced dissociation-tandem mass spectrometry using the ‘bottom-up’ mass spectrometric approach. As a result of the above teachings, the sequences of certain diagnostic Tryptic peptides have been elucidated. It has also been determined that Atlantic cod Vitellogenin shares a series of common peptides with the two different known Vitellogenin sequences of Haddock, a closely related species. Thus, it is likely that Atlantic cod might also co-express at least two distinct forms of vitellogenin.

The use of the common signature peptide for a plurality of species is a significant advantage in regions where both species play an important role in commercial aquaculture ventures. Furthermore, using the teachings of the present invention, one skilled in the art can readily determine other fish species which share this common tryptic peptide.

This rapid and facile technique of the present invention can thus be considered as a viable alternative to known conventional immunoassays, like ELISA and RIA. The ‘signature peptide’ approach of the present invention eliminates the intensive work required for the development and production of specific polyclonal and monoclonal antibodies. Moreover, the ‘signature peptide’ method of the present invention can benefit from the use of a laboratory synthesized high quality homogeneous standard peptide, and, thus overcome the difficulty of purifying the complex phosphoglycolipoprotein from fish blood. Additionally, due to the declining trend in the use of RIA due to radioactivity hazards, higher regulatory costs and significant disposal problems caused by radiochemicals, the present invention has significant advantages in replacing such known techniques.

In contrast to antibody affinity in immunoassays, the specificity of the method of the present invention simply relies on the distinctiveness of the signature peptide and the power of tandem mass spectrometry to detect it. Moreover, it is considered likely that the method of the present invention will be less susceptible to the false positive results sometimes encountered in immunological assays.

Another benefit of the described technique is the low volume (5 μL) of plasma required for analyses. Although blood extraction is definitely an invasive but non-destructive procedure for biomarker analysis⁶³, the small volume used allows the fish to be returned to their habitat with minimal stress impact.

Finally, the present invention contemplates what units should be used for quantification of proteins. This issue, general to quantification of any protein, was significantly pronounced for Vtg because its molecular weight can vary within the same species or among species according to the degree of post translational modifications and its lipid content. The approach described herein permits concentrations to be easily expressed as moles (or fractions) per volume units, supposing the peptide-to-protein molar relation is known. Thus, the concentrations obtained are more convenient for comparing purposes such as in studies that analyze interspecies or intra-species Vtg concentration variations.

It will be understood that various modifications can be made to the above-described preferred embodiments without departing from the spirit and scope of the invention described herein.

### REFERENCE LIST

1. Hara A and Hirai H. Comparative studies on immunochemical properties of female-specific serum protein and egg yolk proteins in rainbow trout (Salmo gairdneri). Comp Biochem. Physiol B. 1978; 59: 339.
2. Tata J R and Smith D F. Vitellogenesis: a versatile model for hormonal regulation of gene expression. Recent Prog. Horm. Res. 1979; 35: 47.
3. Ng, T. B. and Idler, D. R. Yolk formation and differentiation in teleost fishes; In Fish physiology; Hoar, W. S, and Specker, J. L. Academic Press: New York, 1983; 373-404.
4. Pan M L, Bell W J, and Telfer W H. Vitellogenic blood protein synthesis by insect fat body. Science. 1969; 165: 393.
5. Ait-Aissa S, Ausseil O, Palluel O, Vindimian E, Garnier-Laplace J, and Porcher J M. Biomarker responses in juvenile rainbow trout (Oncorhynchus mykiss) after single and combined exposure to low doses of cadmium, zinc, PCB77 and 17beta-oestradiol. Biomarkers. 2003; 8: 491.
6. Denslow N D, Chow M C, Kroll K J, and Green L. Vitellogenin as a Biomarker of Exposure for Estrogen or Estrogen Mimics. Ecotoxicology. 1999; 8: 385.
7. Heppell S A, Denslow N D, Folmar L C, and Sullivan C V. Universal assay of vitellogenin as a biomarker for environmental estrogens. Environ. Health Perspect. 1995; 103 Suppl 7: 9.
8. Sumpter J P and Jobling S. Vitellogenesis as a biomarker for estrogenic contamination of the aquatic environment. Environ. Health Perspect. 1995; 103 Suppl 7: 173.
9. McClain J S, Oris J T, Burton G A, Jr., and Lattier D. Laboratory and field validation of multiple molecular biomarkers of contaminant exposure in rainbow trout (Oncorhynchus mykiss). Environ. Toxicol. Chem. 2003; 22: 361.
10. Banoub J, Thibault P, Mansour A, Cohen A, Heeley D H, and Jackman D. Characterisation of the intact rainbow trout vitellogenin protein and analysis of its derived tryptic and cyanogen bromide peptides by matrix-assisted laser desorption/ionisation time-of-flight-mass spectrometry and electrospray ionisation quadrupole/time-of-flight mass spectrometry. Eur. J. Mass Spectrom. 2003; 9: 509.
11. Banoub J, Cohen A, Mansour A, and Thibault P. Characterization and de novo sequencing of Atlantic salmon vitellogenin protein by electrospray tandem and matrix-assisted laser desorption/ionization mass spectrometry. Eur. J. Mass Spectrom. 2004; 10: 121.
12. Cohen A M, Mansour A A, and Banoub J H. ‘De novo’ sequencing of Atlantic cod vitellogenin tryptic peptides by matrix-assisted laser desorption/ionization quadrupole time-of-flight tandem mass spectrometry: similarities with haddock vitellogenin. Rapid Commun. Mass Spectrom. 2005; 19: 2454.
13. Wallace R. A. and Jared D. W. Studies on amphibian yolk. VII. Serum phosphoprotein synthesis by vitellogenic females and estrogen-treated males of Xenopus laevis. Can J. Biochem. 1968; 46: 953.
14. Palmer B D, Huth L K, Pieto D L, and Selcer K W. Vitellogenin as a biomarker for xenobiotic estrogens in an amphibian model system. Environmental Toxicology and Chemistry. 1998; 17: 30.
15. Tyler C R, van Aerle R, Nilsen M V, Blackwell R, Maddix S, Nilsen B M, Berg K, Hutchinson T H, and Goksoyr A. Monoclonal antibody enzyme-linked immunosorbent assay to quantify vitellogenin for studies on environmental estrogens in the rainbow trout (Oncorhynchus mykiss). Environ. Toxicol. Chem. 2002; 21: 47.
16. Campbell C M and Idler D R. Characterization of an estradiol-induced protein from rainbow trout serum as vitellogenin by the composition and radioimmunological cross reactivity to ovarian yolk fractions. Biol. Reprod. 1980; 22: 605.
17. Norberg B and Haux C. An homologous radioimmunoassay for brown trout (Salmo trutta) vitellogenin. Fish Physiology and Biochemistry [FISH PHYSIOL. BIOCHEM.]. 1988; 5: 59.
18. So Y P, Idler D R, and Hwang S J. Plasma vitellogenin in landlocked Atlantic salmon (Salmo salar Ouananiche): isolation, homologous radioimmunoassay and immunological cross-reactivity with vitellogenin from other teleosts. Comp Biochem. Physiol B. 1985; 81: 63.
19. Bowman C J and Denslow N D. Development and Validation of a Species- and Gene-Specific Molecular Biomarker: Vitellogenin mRNA in Largemouth Bass (Micropterus salmoides). Ecotoxicology [Ecotoxicology]. vol. 8. 1999; 88: 399.
20. Zhang H, Yan W, and Aebersold R. Chemical probes and tandem mass spectrometry: a strategy for the quantitative analysis of proteomes and subproteomes. Curr. Opin. Chem. Biol. 2004; 8: 66.
21. Riggs L, Sioma C, and Regnier F E. Automated signature peptide approach for proteomics. J. Chromatogr. A. 2001; 924: 359.
22. Ji J, Chakraborty A, Geng M, Zhang X, Amini A, Bina M, and Regnier F. Strategy for qualitative and quantitative analysis in proteomics based on signature peptides. J. Chromatogr. B Biomed. Sci. Appl. 2000; 745: 197.
23. Geng M, Ji J, and Regnier F E. Signature-peptide approach to detecting proteins in complex mixtures. J. Chromatogr. A. 2000; 870: 295.
24. Gygi S P, Rist B, Gerber S A, Turecek F, Gelb M H, and Aebersold R. Quantitative analysis of complex protein mixtures using isotope-coded affinity tags. Nat. Biotechnol 1999; 17: 994.
25. Gygi SP, Rist B, Griffin T J, Eng J, and Aebersold R. Proteome analysis of low-abundance proteins using multidimensional chromatography and isotope-coded affinity tags. J. Proteome. Res. 2002; 1: 47.
26. Yu L R, Conrads T P, Uo T, Issaq H J, Morrison R S, and Veenstra T D. Evaluation of the acid-cleavable isotope-coded affinity tag reagents: application to camptothecin-treated cortical neurons. J. Proteome. Res. 2004; 3: 469.
27. Ong S E, Blagoev B, Kratchmarova I, Kristensen D B, Steen H, Pandey A, and Mann M. Stable isotope labeling by amino acids in cell culture, SILAC, as a simple and accurate approach to expression proteomics. Mol. Cell. Proteomics. 2002; 1: 376.
28. DeSouza L, Diehl G, Rodrigues M J, Guo J, Romaschin A D, Colgan T J, and Siu K W. Search for cancer markers from endometrial tissues using differentially labeled tags iTRAQ and cICAT with multidimensional liquid chromatography and tandem mass spectrometry. J Proteome. Res. 2005; 4: 377.
29. Yao X, Freas A, Ramirez J, Demirev P A, and Fenselau C. Proteolytic 18O labeling for comparative proteomics: model studies with two serotypes of adenovirus. Anal. Chem. 2001; 73: 2836.
30. Yao X, Afonso C, and Fenselau C. Dissection of proteolytic 18O labeling: endoprotease-catalyzed 16O-to-18O exchange of truncated peptide substrates. J Proteome. Res. 2003; 2: 147.
31. Masselon C, Anderson G A, Harkewicz R, Bruce J E, Pasa-Tolic L, and Smith R D. Accurate mass multiplexed tandem mass spectrometry for high-throughput polypeptide identification from mixtures. Anal. Chem. 2000; 72: 1918.
32. Smith R D, Anderson G A, Lipton M S, Pasa-Tolic L, Shen Y, Conrads T P, Veenstra T D, and Udseth H R. An accurate mass tag strategy for quantitative and high-throughput proteome measurements. Proteomics. 2002; 2: 513.
33. Wang W, Zhou H, Lin H, Roy S, Shaler T A, Hill L R, Norton S, Kumar P, Anderle M, and Becker C H. Quantification of proteins and metabolites by mass spectrometry without isotopic labeling or spiked standards. Anal. Chem. 2003; 75: 4818.
34. Qian W J, Monroe M E, Liu T, Jacobs J M, Anderson G A, Shen Y, Moore R J, Anderson D J, Zhang R, Calvano S E, Lowry S F, Xiao W, Moldawer L L, Davis R W, Tompkins R G, Camp D G, and Smith R D. Quantitative proteome analysis of human plasma following in vivo lipopolysaccharide administration using 16O/18O labeling and the accurate mass and time tag approach. Mol. Cell Proteomics. 2005; 4: 700.
35. Conrads T P, Anderson G A, Veenstra T D, Pasa-Tolic L, and Smith R D. Utility of accurate mass tags for proteome-wide protein identification. Anal. Chem. 2000; 72: 3349.
36. Pasa-Tolic L, Masselon C, Barry R C, Shen Y, and Smith R D. Proteomic analyses using an accurate mass and time tag strategy. Biotechniques. 2004; 37: 621.
37. Stritmatter E F, Ferguson P L, Tang K, and Smith R D. Proteome analyses using accurate mass and elution time peptide tags with capillary LC time-of-flight mass spectrometry. J Am. Soc. Mass Spectrom. 2003; 14: 980.
38. Silva J C, Denny R, Dorschel C A, Gorenstein M, Kass I J, Li G Z, McKenna T, Nold M J, Richardson K, Young P, and Geromanos S. Quantitative proteomic analysis by accurate mass retention time pairs. Anal. Chem. 2005; 77: 2187.
39. Elias D A, Monroe M E, Marshall M J, Romine M F, Belieav A S, Fredrickson J K, Anderson G A, Smith R D, and Lipton M S. Global detection and characterization of hypothetical proteins in Shewanella oneidensis MR-1 using LC-MS based proteomics. Proteomics. 2005; 5: 3120.
40. Zhang F, Bartels M J, Brodeur J C, and Woodburn K B. Quantitative measurement of fathead minnow vitellogenin by liquid chromatography combined with tandem mass spectrometry using a signature peptide of vitellogenin. Environ. Toxicol. Chem. 2004; 23:1408.
41. Barnidge D R, Dratz E A, Martin T, Bonilla L E, Moran L B, and Lindall A. Absolute quantification of the G protein-coupled receptor rhodopsin by LC/MS/MS using proteolysis product peptides and synthetic peptide standards. Anal. Chem. 2003; 75: 445.
42. Barr J R, Maggio V L, Patterson D G, Jr., Cooper G R, Henderson L O, Turner W E, Smith S J, Hannon W H, Needham L L, and Sampson E J. Isotope dilution—mass spectrometric quantification of specific proteins: model application with apolipoprotein A-I. Clin. Chem. 1996; 42: 1676.
43. Jeppsson J O, Kobold U, Barr J, Finke A, Hoelzel W, Hoshino T, Miedema K, Mosca A, Mauri P, Paroni R, Thienpont L, Umemoto M, and Weykamp C. Approved IFCC reference method for the measurement of HbA1c in human blood. Clin. Chem. Lab Med. 2002; 40: 78.
44. Gerber S A, Rush J, Stemman O, Kirschner M W, and Gygi S P. Absolute quantification of proteins and phosphoproteins from cell lysates by tandem MS Proc. Natl. Acad. Sci. U.S.A. 2003; 100: 6940.
45. Kirkpatrick D S, Gerber S A, and Gygi S P. The absolute quantification strategy: a general procedure for the quantification of proteins and post-translational modifications. Methods. 2005; 35: 265.
46. Gygi S P and Aebersold R. Absolute quantitation of 2-D protein spots. Methods Mol Biol 1999; 112: 417.
47. Anderson N L, Anderson N G, Haines L R, Hardie D B, Olafson R W, and Pearson T W. Mass spectrometric quantitation of peptides and proteins using Stable Isotope Standards and Capture by Anti-Peptide Antibodies (SISCAPA). J Proteome. Res. 2004; 3: 235.
48. Wunschel D, Schultz I, Skillman A, and Wahl K. Method for detection and quantitation of fathead minnow vitellogenin (Vtg) by liquid chromatography and matrix-assisted laser desorption/ionization mass spectrometry. Aquat. Toxicol 2005; 73: 256.
49. Chernushevich I V, Loboda A V, and Thomson B A. An introduction to quadrupole-time-of-flight mass spectrometry. J. Mass Spectrom. 2001; 36: 849.
50. Chernushevich IV Duty cycle improvement for a quadrupole-time-of-flight mass spectrometer and its use for precursor ion scans. Eur. J. Mass Spectrom. 2000; 6:471.
51. Jeanville P M, Woods J H, Baird T J, III, and Estape E S. Direct determination of ecgonine methyl ester and cocaine in rat plasma, utilizing on-line sample extraction coupled with rapid chromatography/quadrupole orthogonal acceleration time-of-flight detection. J Pharm. Biomed. Anal. 2000; 23: 897.
52. Luo Y, Rudy J A, Uboh C E, Soma L R, Guan F, Enright J M, and Tsang D S. Quantification and confirmation of flunixin in equine plasma by liquid chromatography-quadrupole time-of-flight tandem mass spectrometry. J Chromatogr. B Analyt. Technol. Biomed. Life Sci. 2004; 801: 173.
53. Marvin L F, Delatour T, Tavazzi I, Fay L B, Cupp C, and Guy P A. Quantification of o,o′-dityrosine, o-nitrotyrosine, and o-tyrosine in cat urine samples by LC/electrospray ionization-MS/MS using isotope dilution. Anal. Chem. 2003; 75: 261.
54. Michalet S, Favreau P, and Stocklin R. Profiling and in vivo quantification of proteins by high resolution mass spectrometry: the example of goserelin, an analogue of luteinizing hormone-releasing hormone. Clin. Chem. Lab Med. 2003; 41: 1589.
55. Biemann K Sequencing of peptides by tandem mass spectrometry and high-energy collision-induced dissociation. Methods Enzymol. 1990; 193: 455.
56. Cejka J, Vodrazka Z, and Salak J. Carbamylation of globin in electrophoresis and chromatography in the presence of urea. Biochim. Biophys. Acta. 1968; 154: 589.
57. Makler M T Carbamylation of human HbF on carboxy methyl cellulose column in 8 M urea pH 6.7. Biochem Biophys. Res. Commun. 1980; 94: 967.
58. Lippincott J and Apostol I. Carbamylation of cysteine: a potential artifact in peptide mapping of hemoglobins in the presence of urea. Anal. Biochem. 1999; 267: 57.
59. Silversand C, Hyllner S J, and Haux C. Isolation, immunochemical detection, and observations of the instability of vitellogenin from four teleosts. Journal of Experimental Zoology. 1993; 267: 587.
60. Copeland P A, Sumpter J P, Walker T K, and Croft M. Vitellogenin levels in male and female rainbow trout (Salmo gairdneri Richardson) at various stages of the reproductive cycle. Comp Biochem. Physiol B. 1986; 83: 487.
61. Bon E, Barbe U, Nunez R J, Cuisset B, Pelissero C, Sumpter J P, and Le Menn F. Plasma vitellogenin levels during the annual reproductive cycle of the female rainbow trout (Oncorhynchus mykiss): establishment and validation of an ELISA. Comp Biochem Physiol B Biochem Mol. Biol. 1997; 117: 75.
62. Nilsen B M, Berg K, Eidem J K, Kristiansen S I, Brion F, Porcher J M, and Goksoyr A. Development of quantitative vitellogenin-ELISAs for fish test species used in endocrine disruptor screening. Anal. Bioanal. Chem. 2004; 378: 621.
63. Casin S, Fossi M C, Cavallaro K, Marsili L, and Lorenzani J. The use of porphyrins as a non-destructive biomarker of exposure to contaminants in two sea lion populations. Mar. Environ. Res. 2002; 54: 769.
64. Reith M, Munholland J, Kelly J, Finn R N, and Fyhn H J. J. Exp. Zool. 2001; 291: 58.
65. Kinter, M. and Sherman, N. E. Protein sequencing and identification using tandem mass spectrometry, John Wiley & Sons: New York, 2000.
66. Nelson, J. S. Fishes of the World, 2 ed.; John Wiley & Sons: New York, 1984.
67. Specker, J. L. and Sullivan, C. V. Vitellogenesis in fish: Satus and Perspectives; In Perspectives in comparative endocrinology; Davey, K. G., Peter, R. G., and Tobe, S. S. National Research Council of Canada: Ottawa, 1994; 304-315.

What is claimed is:

1. A method of quantification of fish plasma Vitellogenin using signature peptides in a mass spectrometry comprising the steps of selecting a desired signature peptide from at least one fish species, and determining the presence or absence of the selected signature peptide in said fish species by liquid chromatography in combination with mass spectrometry.

2. The method as defined in claim 1, in which at least two fish species are employed, each species having a common signature peptide with the other.

3. The method as defined in claim 1, wherein two fish species comprising trout and salmon are employed.

4. The method as defined in claim 1, wherein the two fish species are Atlantic cod and haddock.

5. The method as defined in claim 1, wherein the mass spectrometry is carried out using a hybrid tandem mass spectrometer.

6. The method as defined in claim 1, wherein the signature peptide has the following sequence:
Thr-Tyr-Phe-Ala-Gly-Ala-Ala-Ala-Asp-Val-Leu-Glu-
Val-Gly-Val-Arg.

7. The method as defined in claim 1, wherein the signature peptide is DLGLAYTEK.

8. The method as defined in claim 1, wherein the signature peptide has the following sequence:
Phe Phe Gly Gln Glu Ile Ala Asn Ile Asp Lys.

9. The method as defined in claim 1, wherein the signature peptide has the following sequence:
Glu Ile Val Leu Leu Gly Tyr Gly Thr Met Ile Ser
Lys.

10. The method as defined in claim 1, wherein the signature peptide has the following sequence:
Tyr Glu Ser Phe Ala Val Ala Arg.

11. A signature peptide selected from the group consisting of peptides having the following sequences:
Tyr-Tyr-Phe-Ala-Gly-Ala-Ala-Ala-Asp-Val-Leu-Glu-
Val-Gly-Val-Arg, Asp Leu Gly Leu Ala Tyr Thr Glu
Lys, Phe Phe Gly Gln Glu Ile Ala Asn Ile Asp Lys,
Glu Ile Val Leu Leu Gly Tyr Gly Thr Met Ile Ser
Lys, and Tyr Glu Ser Phe Ala Val Ala Arg.

12. A kit for testing quantification of fish plasma vitellogenin having a signature peptide.

13. The kit according to claim 12 wherein the signature peptide is
Thr-Tyr-Phe-Ala-Gly-Aly-Ala-Ala-Asp-Val-Leu-Glu-
Val-Gly-Val-Arg-or Asp Leu Gly Leu Ala Tyr Thr Glu
Lys.

14. The kit according to claim 12, wherein the signature peptide is Phe Phe
Gly Gln Glu Ile Ala Asn Ile Asp Lys, Glu Ile Val
Leu Leu Gly Tyr Gly Thr Met Ile Ser Lys or Tyr Glu
Ser Phe Ala Val Ala Arg.
